(19) United States
(12) Patent Application Publication
(10) Pub. No.: US 2010/0035848 A1
(43) Pub. Date: Feb. 11, 2010

(54) THERAPY FOR DISORDERS OF THE PROXIMAL DIGESTIVE TRACT

(76) Inventors: Stephen Richard Donahue, Lexington, MA (US); Michael David Jacobson, Cambridge, MA (US); John Joseph Byrnes, III, Ashland, MA (US); Luz-Maria Guzman, Watertown, MA (US); Thomas Michael Barnes, Brookline, MA (US); Robert Mark Coopersmith, Chestnut Hill, MA (US); Scott Edward Malstrom, Reading, MA (US)

Correspondence Address: HARNESS, DICKEY, & PIERCE, P.L.C, 7700 Bonhomme, Suite 400, ST. LOUIS, MO, 63105, US

(21) Appl. No.: 12/401,143

(22) Filed: Mar. 10, 2009

Related U.S. Application Data
(60) Provisional application No. 61/035,271, filed on Mar. 10, 2008.

(51) Int. Cl. A61K 31/4164; A61P 29/00; A61P 1/00; A61P 1/04; A61K 31/60; A61K 31/421; A61K 31/5415

(52) U.S. Cl. 514/161; 514/400; 514/374; 514/226.5

(57) ABSTRACT

A method for managing or treating an inflammatory, erosive, dyspeptic or reflux disorder of the proximal digestive tract of a subject comprises administering to the subject a therapeutically effective amount of an ACE2 inhibitor. A therapeutic combination, useful to treat any disease or condition in which an NSAID is indicated, comprises an NSAID in an anti-inflammatory, analgesic or antipyretic effective amount and a gastroprotective agent that comprises an ACE2 inhibitor in an amount effective to protect mucosal surfaces of the proximal digestive tract from erosion or ulceration induced by the NSAID.

This application claims the benefit of U.S. provisional patent application Ser. No. 61/035,271, filed on Mar. 10, 2008, the disclosure of which is incorporated by reference herein in its entirety.

This application contains subject matter that is related to subject matter of co-assigned U.S. application Ser. No. 11/851,669 and co-assigned U.S. application Ser. No. 11/851,694, both filed on Sep. 7, 2007, the disclosure of each of which is incorporated by reference herein in its entirety.

### FIELD OF THE INVENTION

The present invention relates to pharmacotherapy for inflammatory, erosive, dyspeptic and reflux disorders of the proximal digestive tract including esophagus, stomach and duodenum. More particularly, the invention relates to methods for managing or treating such disorders and for protecting mucosal surfaces of the proximal digestive tract from erosion or ulceration.

### BACKGROUND

The digestive tract, also referred to as the alimentary canal (nourishment canal) or the gut, is part of the digestive system, i.e., the system of organs within multicellar animals which takes in food, digests it to extract energy and nutrients, and expels the remaining waste. This process is called digestion. As defined herein, the digestive tract includes those organs through which food or solid exereta pass in the course of the digestive process, but excludes those organs of the digestive system, adjacent to and connecting with the digestive tract, that store and/or secrete substances aiding in digestion, for example liver, gallbladder and pancreas.

The term “proximal digestive tract” herein means that part of the digestive tract extending from esophagus to duodenum, comprising the esophagus (including the cardiac antrum and esophageal sphincter), stomach (including the cardia, corpus, pyloric antrum, pyloric canal, pylorus and pyloric sphincter) and duodenum (the proximal portion of the small intestine, including the duodenal bulb). The esophagus, stomach and duodenum have a mucosal lining (esophageal, gastric and duodenal mucosa respectively).

Disorders of the proximal digestive tract can be acute or chronic, and include inflammatory, erosive, dyspeptic and reflux disorders. These are not discrete classes of disorder, hut overlap one another and in some cases have symptoms and/or etiological factors in common.

In inflammatory disorders of the proximal digestive tract, the mucosal lining typically becomes inflamed. Inflammation can be general to all or a major portion of the proximal digestive tract (and can extend to distal portions of the tract including jejunum, ileum and large intestine), or relatively localized, for example in the esophagus (esophagitis), stomach (gastritis), pyloric antrum (antral gastritis), pylorus (pyloritis) or duodenum (duodenitis). Such inflammatory disorders can be acute or chronic and can arise from a variety of identifiable causes or can, in some cases, be idiopathic. The most common causal agent, especially in gastritis (including antral gastritis and pyloritis) and duodenitis, is bacterial infection, the principal infective agent being the gram-negative bacillus Helicobacter pylori. Irritant agents, including irritants in food, beverages and medications, can also precipitate inflammation. Especially common and troublesome are inflammatory conditions induced by nonsteroidal anti-inflammatory drugs (NSAIDs), which are believed to disrupt the mucosal layer by inhibiting cyclooxygenase activity, resulting in reduced levels of protective prostaglandins; other irritants that can lead to inflammation of the proximal digestive tract include alcohol and caffeine. In reflux gastritis, inflammation results from or is exacerbated by reflux of bile and/or pancreatic fluids from the duodenum into the stomach. In reflux esophagitis, inflammation results from or is exacerbated by reflux of gastric fluids from the stomach to the esophagus. Yet other causes of esophageal, gastric and duodenal inflammatory disorders include autoimmune disease, gastroduodenal Crohn's disease and Zollinger-Ellison syndrome.

A particular inflammatory disorder involving the duodenum and, usually to a lesser extent, the distal portions of the small intestine (jejunum and ileum) is sprue, also known as celiac sprue or celiac disease. In sprue, inflammation of the mucosa is caused by a cascade of immune events, and is activated in predisposed individuals by exposure to certain proteins called gliadins (of which gluten is the most important) in cereal grains, particularly wheat, barley and oats.

Inflammatory involvement has been identified in some cases of gastroparesis, a condition in which reduced muscle function prevents normal stomach emptying.

Inflammation of the mucosal lining of the proximal digestive tract can lead to atrophy of the mucosa and parietal cells, particularly in the stomach (atrophic gastritis). Loss of gastric parietal cells, particularly in gastritis of autoimmune origin, results in absence of a glycoprotein known as intrinsic factor essential for absorption of vitamin B₁₂, leading to vitamin B₁₂ deficiency, manifesting as pernicious anemia.

More commonly, inflammation progresses to erosion or ulceration of the wall of the proximal digestive tract. Peptic ulcer disease is a term generally given to erosive or ulcerative inflammation of the stomach and/or duodenum in which the normal mucosal protection from injurious effects of gastric fluid principally gastric acid and pepsin) is diminished. Any of the causes of inflammation mentioned above can be involved, but again the most common causes include H. pylori infection and NSAID use. It has been estimated that 10-30% of patients on regular NSAID therapy develop gastric ulcers. Peptic ulcer disease can be further aggravated by various factors including diet, stress and tobacco use, and can be secondary to other diseases including chronic kidney disease, chronic obstructive pulmonary disease (COPD) and alcoholism. Peptic ulcers can bleed (hemorrhagic gastritis, hemorrhagic duodenitis) and in extreme cases can perforate the stomach or duodenal wall with potentially severe or life-threatening consequences including peritonitis and sepsis.

The term dyspepsia refers to symptoms originating in the proximal digestive tract, including pain and discomfort often with feelings of early satiety, bloating, eructation, dysphagia, nausea and/or vomiting. The most common form of pain arising from dyspepsia is heartburn. The term “dyspeptic disorder” is used herein to refer to any disease or condition in which such symptoms are manifested, including inflammatory and/or erosive diseases as described above and gastroesophageal reflux disease (GERD), described below. Peptic ulcers and GERD are especially well-known etiological factors in dyspepsia. However, inflammatory, erosive and reflux disorders do not necessarily give rise to dyspepsia; furthermore, dyspepsia can arise without involvement of inflammatory, erosive or reflux disorders. Indeed, the most common type of dyspeptic disorder is one having no identifiable organic origin; this type is variously referred to as functional dyspepsia, nonulcer dyspepsia or idiopathic dyspepsia. Postprandial distress syndrome is a form of functional dyspepsia in which symptoms including early satiety occur after eating. In epigastric pain syndrome, symptoms including pain are more constant and less meal-related.

GERD involves reflux of gastric acids into the esophagus, sometimes all the way to the mouth (acid regurgitation). GERD can be acute or, more commonly, chronic. Symptoms, including heartburn and acid regurgitation, tend to be exacerbated by ingestion of fatty foods and caffeine, and by recumbent position, Damage to the mucosal lining of the esophagus (reflux esophagitis) occurs in many cases, often leading to development of esophageal erosions or ulcers (erosive esophagitis). Such damage is visible by endoscopy and, if unchecked, can in some cases lead to a precancerous condition known as Barrett's esophagus and thence to esophageal cancer. Gastroesophageal reflux in the absence of visible injury to the esophageal mucosa is known as endoscopy-negative reflux disease or nonerosive reflux disease (NERD).

Disorders of the proximal digestive tract mentioned above are extraordinarily common and widespread. For example, Schwartz (2002) Western J. Med. 176:98-103 provides the following statistics:
26% of the adult population complains of dyspepsia each year;
10% of the adult population experiences heartburn daily;
there were more than 18 million ambulatory care visits in the U.S. in 1997 for stomach and abdominal pain, cramps and spasms;
dyspepsia was the third most common symptomatic reason for ambulatory care visits in 1997;
U.S. prevalence of peptic ulcer disease is 1%;
lifetime cumulative incidence of peptic ulcer disease is 10% for men and 4% for women;
400,000 new cases of peptic ulcer disease are diagnosed each year;
duodenal ulcers are twice as common in men as in women;
duodenal ulcers are 1.5 times as common as gastric ulcers;
peak incidence of peptic ulcer disease is in the 40-49 age group for men and the 50-69 age group for women;
90% of patients with duodenal ulcers have recurrence if not given maintenance therapy and H. pylori is not eradicated;
prevalence of H. pylori colonization in the general population is 40% in the U.S. and 70-80% in the developing world;
in the U.S., prevalence of H. pylori is 50% by age 50 and is highest in African-American and Latino populations;
most current infections reflect exposure to H. pylori during childhood; with improved sanitation new infection rates are less than 0.5% per year;
prevalence of H. pylori infection in functional dyspepsia patients is no higher than in the general population;
10-15% of chronic GERD patients develop Barrett's esophagus.

Bazaldua & Schneider (1999) American Family Physician 60:1773-1788 further report that:
up to 60% of dyspepsia cases have no identified structural or biochemical etiology (functional dyspepsia);
15-25% of dyspepsia cases are associated with peptic ulcer disease;
5-15% of dyspepsia cases are associated with reflux esophagitis.

Where H. pylori infection is an etiological factor (i.e., commonly in duodenitis, gastritis, peptic ulcer disease and dyspepsia related to these, but not in GERD or most cases of functional dyspepsia), treatment of proximal digestive tract disorders typically includes medication with one or more antibiotics. To help combat emergence of antibiotic resistance, at least two antibiotics are usually prescribed, for example (a) metronidazole or clarithromycin and (b) tetracycline or amoxicillin.

Whether or not H. pylori infection is involved, the treatment regimen typically includes an antisecretory agent to inhibit gastric acid secretion. These agents are of two main classes: proton-pump inhibitors (PPIs) such as omeprazole, lansoprazole or rabeprazole; and histamine H₂-receptor antagonists such as cimetidine, famotidine and ranitidine. Older medications that neutralize excess acid in the stomach (antacids) can still be useful adjuncts. Other antiulcer medications include sucralfate and prostaglandins such as misoprostol.

McColl (2000) Gastroenterol. 35(Suppl. 12):47-50 reported that H. pylori-negative ulcers (for example NSAID-induced or idiopathic gastroduodenal ulcers) are more difficult to control with antisecretory drugs than H. pylori-positive ulcers.

Inflammatory, erosive, dyspeptic and reflux disorders of the proximal digestive tract have a major impact on human wellbeing, on health care costs and on lost work time and productivity. Existing therapies including those mentioned above are effective in many but not all patients. For disorders of such prevalence and diversity there remains a need for new and alternative treatments.

The nuclear factor κB (NF-κB) signaling pathway is involved in a wide range of pro-inflammatory effects. See, e.g., Schreiber et al. (1998) Gut 42; 477-484. Augiotensin II (Ang II), a member of the renin-angiotensin system (RAS) and the primary product of angiotensin-converting enzyme (ACE), is known to exert pro-inflammatory effects in a variety of tissues, via its type 1 and type 2 receptors (AT₁ and AT₂ respectively) and, in many cases, ultimately through activation of NE-κB, as indicated below.

In the classical pathway of Ang II synthesis in the circulating RAS, the precursor of Ang II is angiotensinogen, which is principally produced in the liver and then cleaved by renin to form angiotensin I (Ang I), which is converted by ACE into Ang II that is carried to various target cells via the circulatory system. See, e.g., Inokuchi et al. (2005) Gut 54:349-356, and sources cited therein. In addition, tissue-specific renin-angiotensin systems have been identified in many organs, suggesting that various tissues have the ability to synthesize Ang II independently of circulating RAS, including kidney, brain, aorta, adrenal gland, heart, stomach and colon.

Donoghue et al. (2000) Circ. Res. 87:1-9 reported identification of a carboxy-peptidase related to ACE from sequencing of a human heart failure ventricle cDNA library. This carboxypeptidase, ACE2, was stated to be the first known human homolog of ACE. The authors further reported that the metalloprotease catalytic domains of ACE2 and ACE are 42% identical, and that, in contrast to the more ubiquitous ACE, ACES transcripts are found only in heart, kidney, and testis in the 23 human tissues examined.

U.S. Pat. No. 6,194,556 to Acton et al. discloses novel genes encoding ACE2. Therapeutics, diagnostics and screening assays based on these genes are also disclosed.

Harmer et al. (2002) FEBS Lett. 532:107-110 reported quantitative mapping of the transcriptional expression profile of ACE2 (and the two isoforms of ACE) in 72 human tissues. The study reportedly confined that ACE2 expression is high in renal and cardiovascular tissues. It was further reported that ACE2 shows comparably high levels of expression in the gastrointestinal system, in particular in ileum, duodenum, jejunum, cecum and colon. The authors proposed that in probing functional significance of ACE2, some consideration should be given to a role in gastrointestinal physiology and pathophysiology. Rice et al. (2003) Bull. Br. Soc. Cardiovasc. Res. 16(2):5-11 reviewed potential functional roles of ACE2 and indicated that its expression is mainly localized in testis, kidney, heart and intestines.

Ferreira & Santos (2005) Braz. J. Med. Biol. Res. 38:499-507 have summarized important pathways of the RAS, including roles of ACE and ACE2, as shown in FIG. 1 herein.

As evidence of implication of Ang II, the main product of ACE, in a variety of pro-inflammatory effects, see for example:
Phillips & Kagiyama (2002) Curr. Opin. Investig. Drugs 3(4):569-577, who reviewed literature showing Ang 11 to be a key factor, via NM-κB activation, in promoting inflammation, inter alia, in atherosclerosis;
Costanzo et al. (2003) J. Cell Physiol. 195(3):402-410, who reported up-regulation by Ang 11 of endothelial cell adhesion molecules involved in atherosclerosis, via inflammatory cytokines through NF-κB activation;
Sanz-Rosa et al. (2005) Am. J. Physiol. Heart Circ. Physiol, 288:H1111-H115, who reported that blocking the AT₁ receptor reduces the level of vascular and circulating inflammatory mediators such as NF-κB and tumor necrosis factor-α (TNF-α) in spontaneous hypertension;
Esteban et al. (2004) J. Am. Soc. Nephrol. 15:1514-1529, who reported that Ang II, via AT₁ and AT₂, activates NF-κB and thereby promotes inflammation in obstructed kidney; and
Inokuchi et al. (2005), supra, who reported that in angiotensinogen gene knockout mice, which have low levels of Ang II, inflammatory colitis induced by 2,4,6-trinitrobenzenesulfonic acid (TNBS) is ameliorated, and that blocking the AT₁ receptor also ameliorated TNBS-induced colitis.

Gidaiatov et al. (2000) Klin. Med. (Mosk.) 78(10):40-42 (abstract at www.ncbi. nlm.nih.gov/pubmed/11220899) reported that the ACE inhibitor Renitec (enalapril), administered to treat heart failure, relieved gastralgia and dyspepsia and resulted of healing of duodenal ulcer in 87% of patients treated. Plasma level of “angiotensin” (presumably Ang II as product of ACE) reportedly decreased.

Alekseenko et al. (2004) Klin. Med. (Mosk.) 82(9):42-45 (abstract at www.ncbi. nlm.nih.gov/pubmed/15540422) reported a study in 136 patients receiving the ACE inhibitors enalapril and lisinopril for arterial hypertension. They reported that in patients with concomitant chronic gastritis, administration of ACE inhibitors promoted normalization of proliferative processes in the epitheliun of the gastric mucosa.

Fieichman et al. (2002) Br. J. Clin. Pharmacol. 53:447P-448P reported that in rats gastric epithelial proliferation was increased by Ang II and decreased by drugs that inhibit Ang II formation (the ACE inhibitor enalapril) or antagonize Ang 11 receptors (losartan). A similar effect was reported in human patients treated with enalapril. Of 30 hypertensive patients, the number having gastric mucosal erosions reportedly decreased from 11 to 4 upon treatment with enalapril, and the percentage of patients with dyspeptic symptoms reportedly decreased from 78% to 40%.

Carl-Mcrath et al. (2005) Pathol. Res. & Pract. 201:233, observing that Ang II mediates the decrease in gastric mucosal blood flow seen in gastric ulcers and that ACE inhibitors improve ulcer healing in animal models, investigated expression of ACE in human gastric tissues. They concluded that localization of ACE in the microvasculature of gastric ulcers may account for the ulcer-healing effect of ACE inhibitors and that inhibition of Ang II production may be a therapeutic option in gastritis and gastric ulceration in humans.

The proinflammatory effects of the ACE product Ang 11 have been found to be generally counterbalanced by ACE2 in various studies involving ACE2 disruption and/or mutants lacking the ACE2 gene. See for example:
Crackower et al. (2002) Nature 417(6891):822-828, who reported that disruption of ACE2 or deletion of the ACE2 gene in various rat models raises the level of Ang II;
Huentelman et al. (2005) Exp. Physiol. 90(5):783-790, who reported that injection of a vector encoding ACE2 protects wild-type mice against Ang 11-induced cardiac bypertrophy and fibrosis; and
Imai et al. (2005) Nature 436(7047):112-116, who reported that deletion of the ACE gene or giving ACE2 protein to wild-type mice protects against acid-induced acute lung injury.

The primary product of ACE2, namely angiotensin (1-7), via its receptor (Mas), has generally been found to oppose functions of the ACE product Ang II. See for example:
Guy et al. (2005) Biochim. Biophy. Acta 1751(11):2-8, who reviewed literature indicating inter alia that ACE2 regulates heart and kidney function by control of Ang II levels relative to angiotensin (1-7), and may therefore counterbalance the effects of ACE within the RAS;
Ferreira & Santos (2005), supra, who reviewed literature indicating inter alia that ACE inhibitor benefits may be partly mediated by the ACE2 product angiotensin (1-7), plasma levels of which are greatly increased following chronic administration of ACE inhibitors;
Mendes et al. (2005) Regul. Pept. 125(1-3):29-34, who reported that infusion of angiotensin (1-7) reduces Ang II levels in the heart and postulated that such reduction may contribute to beneficial effects of angiotensin (1-7); and
Tallant & Clark (2003) Hypertension 42:574-579, who reported that angiotensin (1-7) reduces smooth muscle growth after vascular injury, and counteracts stimulation by Ang II of growth and mitogen activated protein (MAP) kinase activity in rat aortic vascular smooth muscle cells.

Thus ACE2 activity appears to counterbalance inflammatory effects of Ang II in a variety of tissues, whether by increasing angiotensin (1-7) levels or reducing Ang II levels or both.

In one scenario, therefore, promotion of ACE2 activity might be of interest for reducing inflammation in inflammatory diseases. Huentelman et al. (2004) Hypertension 44:903-906 proposed, similarly, that in vivo activation of ACE2 could lead to protection and successful treatment for hypertension and other cardiovascular diseases, by counterbalancing the potent vasoconstrictive effects of Ang II.

Agents that inhibit rather than promote ACE2 activity have been described in the art. For example, Huentelman et al. (2004), supra, reported efforts to identify ACE2 inhibitory compounds that inhibit infection by SARS-CoV, the coronavirus responsible for severe acute respiratory syndrome (SARS), for which ACE2 has been found to be a functional receptor. Among the compounds so identified was NAAE (N-(2-aminoethyl)-1-aziridine-ethanamine).

U.S. Pat. No. 6,592,865 to Parry & Sekut is stated in the abstract thereof to relate to use of Ang II in combination with angiotensin 1-9 to potentiate Ang II activity. At column 72, lines 3437 thereof, it is stated: “Since ACE-2 has been found to hydrolyze neurotensin . . . , the invention provides for methods and compositions that can be used for digestive purposes.” There follows, at column 72, line 38—column 75, line 5, an extensive listing of disorders of the digestive system that “the invention” allegedly can be used to treat, prevent or ameliorate.

U.S. Pat. No. 6,900,033 to Parry et al. discloses peptides comprising specific amino acid sequences that are said to specifically bind to ACE2 protein or ACE2-like polypeptides. It is proposed at column 53, lines 63-65 thereof that Can abnormally high a[n]giotensin II level could result from abnormally low activity of ACE-2” and at column 63, lines 21-32 thereof that “ACE-2 binding polypeptides . . . which activate ACE-2-induced signal transduction can be administered to an animal to treat, prevent or ameliorate a disease or disorder associated with aberrant ACE-2 expression, lack of ACE-2 function, aberrant ACE-2 substrate expression, or lack of ACE-2 substrate function. These ACE-2 binding polypeptides may potentiate or activate either all or a subset of the biological activities of ACE-2-mediated substrate action . . . ”. Further, at column 72, lines 40-43 thereof, it is stated: “Since ACE-2 has been found to hydrolyze neurotensin . . . , the invention provides for methods and compositions that can be used for digestive purposes.” There follows, at column 72, line 44—column 75, line 11, an extensive listing of disorders of the digestive system that “the invention” allegedly can be used to treat, prevent or ameliorate. Separately, ACE2 binding peptides that are reported to inhibit ACE2 in vitro are identified in Table 2 at columns 127-130 thereof.

Huang et al. (2003) J. Biol. Chem. 278(18):15532-15540 reported that one such ACE2 inhibitory peptide, namely DX600, exhibited an ACE2 Kᵢ value of 2.8 nM.

Li et al. (2005) Am. J. Physiol. Renal Physiol. 288:F353-F362 reported that DX600 blocked angiotensin I mediated generation of angiotensin (1-7) in rat nephron segments.

U.S. Pat. No. 6,632,830 to Acton et al. discloses compounds comprising a zinc coordinating moiety and an amino acid mimicking moiety, said to be useful for modulating activity of ACE2. More particularly, there are disclosed ACE2 inhibiting compounds of a generic formula presented therein. Such compounds are said to be useful for treating an “ACE-2 associated state” in a patient. “ACE-2 associated states” are said to include high blood pressure and diseases and disorders related thereto, in particular arterial hypertension, congestive heart failure, chronic heart failure, left ventricular hypertropby, acute heart failure, myocardial infarction and cardiomyopathy; states associated with regulating smooth cell proliferation, in particular smooth muscle cell proliferation; kidney diseases and disorders; other hyperadrenergic states; kinetensin associated conditions including those caused by, or contributed to by, abnormal histamine release, for example in local or systemic allergic reactions including eczema, asthma and anaphylactic shock; infertility or other disorders relating to gamete maturation; cognitive disorders; disorders associated with bradykinin and des-Arg bradykinin; and “other examples” (column 36, lines 5867 thereof) that are said to include “SIRS . . . , sepsis, polytrauma, inflammatory bowel disease, acute and chronic pain, bone destruction in rheumatoid and osteo arthritis and periodontal disease, dysmenorrhea, premature labor, brain edema following focal injury, diffise axonal injury, stroke, reperfusion injury and cerebral vasospasm after subarachnoid hemorrhage, allergic disorders including asthma, adult respiratory distress syndrome, wound healing and scar formation.”

Dales et al. (2002) J. Am. Chem. Soc. 124:11852-11853 reported ACE2 IC₅₀ values of a range of such compounds. The most active of these was compound 16, identified therein as having the formula

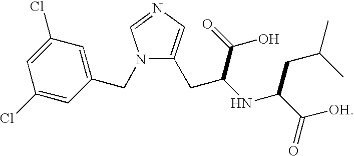

All four stereoisomers of compound 16 were prepared, and the greatest potency was reported for the S,S-isomer, which reportedly had an IC₅₀ for ACE2 of 0.44 nM. The S,S-isomer of the above compound, 2-[1-carboxy-2-[3-(3,5-dichlorobenzyl)-3H-imidazol-4-yl]-ethylamino]-4-methylpentanoic acid, is referred to herein as GL1001 and has previously been referred to as MLN-4760.

U.S. Patent Application Publication No. 2004/0082496 of Acton et al. discloses additional compounds said to be useful for modulating activity of ACE2. Methods of using the inhibitors and pharmaceutical compositions containing the inhibitors to treat a body weight disorder, to decrease appetite, to increase muscle mass, to decrease body fat, to treat diabetes and to treat a state associated with altered lipid metabolism, are also described.

### SUMMARY OF THE INVENTION

In one embodiment, there is now provided a method for managing or treating an inflammatory, erosive, dyspeptic or reflux disorder of the proximal digestive tract of a subject, comprising administering to the subject a therapeutically effective amount of an ACE2 inhibitor. The disorder managed or treated comprises a condition other than chronic gastritis or Crohn's disease, for example at least one of duodenitis, acute gastritis, esophagitis, acute peptic ulcer, functional dyspepsia and gastroesophageal reflux disease (GERD).

In a further embodiment, there is now provided a method for managing or treating an inflammatory, erosive, dyspeptic or reflux disorder of the proximal digestive tract of a subject, comprising administering to the subject a therapeutically effective amount of a compound of formula

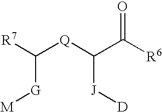

wherein
R⁶ is hydroxyl or a protecting prodrug moiety;
R⁷ is hydrogen, carboxylic acid, ether, alkoxy, an amide, a protecting prodrug moiety, hydroxyl, thiol, heterocyclyl, alkyl or amine;
Q is CH₂, O, NH or NR³, wherein R³ is substituted or unsubstituted C₁₋₅ branched or straight chain alkyl, C₂₋₅ branched or straight chain alkenyl, substituted or unsubstituted acyl, aryl or a C₃₋₈ ring;
G is a covalent bond or a CH₂, ether, thioether, amine or carbonyl liming moiety;
M is heteroaryl, substituted with at least one subanchor moiety comprising a substituted or unsubstituted cycloalkyl or aryl ring, linked thereto through a sublinking moiety (CH₂), or (CH₂)ₙO(CH₂)ₙ where n is an integer from 0 to 3;
J is a bond or a substituted or unsubstituted alkyl, alkenyl or alkynyl moiety; and
D is alkyl, alkenyl, alkynyl, aryl or heteroaryl, optionally linked to G or M to form a ring;
   for example the compound (S,S)-2-[1-carboxy-2-[3-(3,5-dichlorobenzyl)-3H-imidazol-4-yl]-ethylamino]-4-methylpentanoic acid (GL1001); or a pharmaceutically acceptable salt or prodrug thereof. Again, the disorder managed or treated comprises a condition other than chronic gastritis or Crohn's disease, for example at least one of duodenitis, acute gastritis, esophagitis, acute peptic ulcer, functional dyspepsia and GERD.

In a still further embodiment, there is now provided a method for managing or treating an inflammatory, erosive, dyspeptic or reflux disorder that comprises at least one of duodenitis, duodenal ulcer, acute gastritis, acute gastric ulcer, esophagitis, esophageal ulcer, functional dyspepsia and GERD, the method comprising administering to the subject a therapeutically effective amount of an ACE2 inhibitor.

In a still further embodiment, there is now provided a method for managing or treating an inflammatory, erosive, dyspeptic or reflux disorder that comprises at least one of duodenitis, duodenal ulcer, acute gastritis, acute gastric ulcer, esophagitis, esophageal ulcer, functional dyspepsia and GERD, the method comprising administering to the subject a therapeutically effective amount of a compound of formula

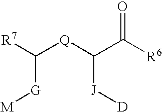

as defined above, for example GL1001, or a pharmaceutically acceptable salt or prodrug thereof.

In a still further embodiment, there is now provided a method for protecting from erosion or ulceration a mucosal surface of the proximal digestive tract of a subject at risk therefor, comprising administering to the subject a therapeutically effective amount of an ACE2 inhibitor. For example, such a method can provide protection from duodenal, gastric and/or esophageal ulcer formation, development or recurrence related to concomitantly administered medication, e.g., comprising a nonsteroidal anti-inflammatory drug (NSAID).

In a still further embodiment, there is now provided a method for protecting from erosion or ulceration a mucosal surface of the proximal digestive tract of a subject at risk therefor, comprising administering to the subject a therapeutically effective amount of a compound of formula

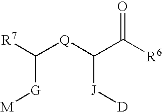

as defined above, for example GL1001, or a pharmaceutically acceptable salt or prodrug thereof. Again, such a method can, for example, provide protection from duodenal, gastric and/or esophageal ulcer formation, development or recurrence related to concomitantly administered medication, e.g., comprising an NSAID.

In a still further embodiment, there is now provided a therapeutic combination comprising an NSAID in an anti-inflammatory, analgesic or antipyretic effective amount and an ACE2 inhibitor in an amount effective to protect mucosal surfaces of the proximal digestive tract from NSAID-induced erosion or ulceration.

In a still further embodiment, there is now provided a therapeutic combination comprising an NSAID in an anti-inflammatory, analgesic or antipyretic effective amount and a gastroprotective agent in an amount effective to protect mucosal surfaces of the proximal digestive tract from NSAID-induced erosion or ulceration. The gastroprotective agent according to this embodiment comprises a compound of formula

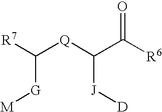

as defined above, for example GL1001, or a pharmaceutically acceptable salt or prodrug thereof.

Other embodiments, including particular aspects of the embodiments summarized above, will be evident from the detailed description that follows.

### BRIEF DESCRIPTION OF THE DRAWINGS

FIG. 1 is a schematic representation of enzymatic pathways of the renin-angiotensin system (RAS) involved in generation of angiotensin peptides. Key:

ACE=angiotensin converting enzyme;

AMP=aminopeptidase;

Ang=angiotensin;

AT₁=angiotensin II type I receptor;

AT₁₋₇=angiotensin (1-7) receptor;

AT₂=angiotensin II type 2 receptor;

D-Amp=dipeptidyl aminopeptidase;

IRAP=insulin regulated aminopeptidase;

NEP=neutral endopeptidase 24.11;

PCP=prolyl carboxypeptidase;

PEP=prolyl endopeptidase.

(From Ferreira & Santos (2005), supra.)

FIG. 2 is a graphical representation of inhibition by GL10001 of in vivo basal NF-κB dependent transcription in recombinant reporter mice, as described in Example 2.

FIG. 3 is a graphical representation of inhibition by GL1001 of in vivo LPS induced NF-κB signaling in mice, as described in Example 3. Mice were pretreated with GL1001 (subcutaneous) for 1 hour before LPS treatment. All mice treated with 0.1 mg/kg LPS (i.v.). Abdominal ROT used for quantitative data (2.76×3.7 cm). Mean±SEM, n=5 for each group; p<0.05, ** p<0.01, ANOVA and student t-test between treatments and controls.

FIG. 4 is a graphical representation of inhibition by GL1001 of in vivo LPS induced NF-κB signaling in mice, as described in Example 3. Male mice were pretreated with GL1001 and LPS, and were imaged, as in FIG. 3. Mean±SEM, n=5 for each group; p<0.05, ** p<0.01, ANOVA and student t-test between treatments and controls.

FIG. 5 is a graphical representation of inhibition by GL1001 of LPS induced NF-κK dependent transcription in selected organs of recombinant reporter mice, as described in Example 3.

### DETAILED DESCRIPTION

According to one embodiment of the invention, various therapeutic methods are described herein, involving administration of an ACES inhibitor to a subject having an inflammatory, erosive, dyspeptic or reflux disorder of the proximal digestive tract.

Any ACE2 inhibitor can be used. In general it will be found useful to select an ACE2 inhibitor having relatively high affinity for ACE2, as expressed for example by IC₅₀ or Ki, whether measured in vitro or in vivo. In one embodiment, the ACE2 inhibitor selected is one that exhibits in vitro an ACE2IC₅₀ and/or an ACE2 Ki not greater than about 1000 nM, for example not greater than about 500 nM, not greater than about 250 nM, or not greater than about 100 nM.

ACE2 inhibitors are known to differ not only in their affinity for ACE2 but also in their selectivity for binding to ACE2 as opposed to the more ubiquitous ACE. In one embodiment, the ACE2 inhibitor exhibits selectivity for ACE2 versus ACE, as expressed by the ratio of IC₅₀(ACE) to IC₅₀(ACE2), of at least about 10², for example at least about 10³, or at least about 10⁴.

Peptide and non-peptide ACE2 inhibitors can be used. Examples of peptide ACE2 inhibitors, and methods for preparing them, can be found for example in above-cited U.S. Pat. No. 6,900,033, which is incorporated herein by reference in its entirety. Peptide compounds exhibiting relatively strong inhibition of ACE2 illustratively include those having peptide sequences identified as DX-512, DX-513, DX-524, DX-525, DX-529, DX-531, DX-599, DX-600, DX-601 and DX-602 in U.S. Pat. No. 6,900,033. Antibodies that bind specifically to the ACE2 protein and thereby inhibit ACE2 activity can also be used in methods and compositions of the present invention.

For many purposes it will be found preferable to use a non-peptide or “small molecule” ACE2 inhibitor. Such compounds tend to be easier to prepare, especially on a large or commercial scale, have lower cost, and present fewer problems in administration and delivery to the active site in the body. In various embodiments, therefore, the ACE2 inhibitor comprises a non-peptide compound or a pharmaceutically acceptable salt thereof or a prodrug thereof.

Illustratively, an ACE2 inhibitor can be of a type disclosed generically in above-cited U.S. Pat. No. 6,632,830, which is incorporated herein by reference in its entirety, including any of the specific compounds disclosed therein along with methods of preparation thereof. In one embodiment, the non-peptide compound comprises a zinc coordinating moiety and an amino acid mimicking moiety.

In one embodiment of the invention, various therapeutic methods are described herein, involving administration of such a non-peptide compound comprises a zinc coordinating moiety and an amino acid mimicking moiety to a subject having an inflammatory, erosive, dyspeptic or reflux disorder of the proximal digestive tract. According to this embodiment, the non-peptide compound can be, but is not necessarily an ACE2 inhibitor. If such compound is an ACE2 inhibitor, it will be understood that any utility or benefit described herein for management or treatment of a proximal digestive tract disorder is not necessarily mediated by ACE2 inhibition. The present embodiment is not limited by any theory of mechanism of action proposed herein

More specifically, the non-peptide compound can have the formula

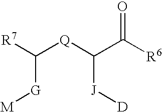

as disclosed in U.S. Pat. No. 6,632,830, wherein
R⁶ is hydroxyl or a protecting prodrug moiety;
R⁷ is hydrogen, carboxylic acid, ether, alkoxy, an amide, a protecting prodrug moiety, hydroxyl, thiol, heterocyclyl, alkyl or amine;
Q is CH₂, O, NH or NR³, wherein R³ is substituted or unsubstituted C₁₋₅ branched or straight chain alkyl, C₂₋₅ branched or straight chain alkenyl, substituted or unsubstituted acyl, aryl or a C₃₋₈ ring;
G is a covalent bond or a CH₂, ether, thioether, amine or carbonyl linking moiety;
M is heteroaryl, substituted with at least one subanchor moiety comprising a substituted or unsubstituted cycloalkyl or aryl ring, linked thereto through a sublinking moiety (CH₂)ₙ or (CH₂)ₙO(CH₂)ₙ where n is an integer from 0 to 3;
J is a bond or a substituted or unsubstituted alkyl, alkenyl or alkynyl moiety; and
D is alkyl, alkenyl, alkynyl, aryl or heteroaryl, optionally linked to G or M to form a ring.

In one embodiment, in the above formula for the non-peptide compound, R⁶ is hydroxyl, R⁷ is carboxylic acid, Q is NH and G is CH₂.

In one embodiment, in the above formula for the non-peptide compound, the heteroaryl group of M is imidazolyl, thienyl, triazolyl, pyrazolyl or thiazolyl. Independently of the selection of heteroaryl group, the subanchor moiety according to this embodiment is C₃₋₆ cycloalkyl, phenyl, methylenedioxyphenyl, naphthalenyl, or phenyl having 1 to 3 substituents independently selected from halo, C₁₋₄ alkyl, C₃₋₆ cycloalkyl, trifluoromethyl, C₁₋₆ alkoxy, trifluoromethoxy, phenyl, cyano, nitro and carboxylic acid groups, and is linked to the heteroaryl group through a (CH₂)ₙ or (CH₂)O(CH₂) sublinking moiety, where n is an integer from 0 to 3.

In one embodiment, in the above formula for the non-peptide compound, J is a bond or CH₂ moiety and D is C₁₋₄ alkyl, C₃₋₄ cycloalkyl or phenyl.

In a more particular embodiment, in the formula for the non-peptide compound:
R⁶ is hydroxyl;
R⁷ is carboxylic acid;
Q is NH;
G is CH₂;
M is imidazolyl, thienyl, triazolyl, pyrazolyl or thiazolyl, linked through a (CH₂)ₙ or (CH₂)O(CH₂) sublinking moiety, where n is an integer from 0 to 3, to a subanchor moiety that is C₃₋₄ cycloalkyl, phenyl, methylenedioxyphenyl, naphthalenyl, or phenyl having 1 to 3 substituents independently selected from halo, C₁₋₆ alkyl, C₃₋₆ cycloalkyl, trifluoromethyl, C₁₋₄ alkoxy, trifluoromethoxy, phenyl, cyano, nitro and carboxylic acid groups;
J is a bond or CH₂ moiety; and
D is C₁₋₄ alkyl, C₃₋₄ cycloalkyl or phenyl.

According to any of the above embodiments the compound can be present in any enantiomeric configuration, e.g., (R,R), (R,S), (S,R) or (S,S), or as a mixture, for example a racemic mixture, of enantiomers. However, in general it is found preferable that the compound be present in the (S,S)-configuration. In one embodiment, the compound is in the (S,S)-configuration and is substantially enantiomerically pure. For example, the compound can exhibit an enantiomeric purity of at least about 90%, at least about 95%, at least about 98% or at least about 99%, by weight of all enantiomeric forms of the compound present.

Illustrative compounds specifically disclosed in U.S. Pat. No. 6,632,830 include the following, each of which can be in any enantiomeric form, illustratively in the (SS)-configuration:
2-[1-carboxy-2-[3-(4-trifluoromethylbenzyl)-3H-imidazol-4-yl]ethylamino]-4-methyl-pentanoic acid;
2-[1-carboxy-2-[3-naphthalen-1-ylmethyl-3H-imidazol-4-yl]ethylamino]-4-methyl-pentanoic acid;
2-[1-carboxy-2-[3-(4-chlorobenzyl)-3H-imidazol-4-yl]ethylamino]-4-methylpentanoic acid;
2-[1-carboxy-2-[3-(3,4-dichlorobenzyl)-3H-imidazol-4-yl]ethylamino]-4-methyl-pentanoic acid;
2-[1-carboxy-2-[3-(4-cyanobenzyl)-3H-imidazol-4-yl]ethylamino]-4-methylpentanoic acid;
2-[1-carboxy-2-[3-(3-chlorobenzyl)-3H-imidazol-4-yl]ethylamino]-4-methylpentanoic acid;
2-[1-carboxy-2-[3-(3,5-dichlorobenzyl)-3H-imidazol-4-yl]ethylamino]-4-methyl-pentanoic acid;
2-[1-carboxy-2-[3-(4-methylbenzyl)-3H-imidazol-4-yl]ethylamino]-4-methylpentanoic acid;
2-[1-carboxy-2-[3-(3,4-dimethylbenzyl)-3H-imidazol-4-yl]ethylamino]-4-methyl-pentanoic acid;
2-[f-carboxy-2-[3-(3-methylbenzyl)-3H-imidazol-4-yl]ethylamino]-4-methylpentanoic acid;
2-[1-carboxy-2-[3-(3,5-dimethylbenzyl)-3H-imidazol-4-yl]ethylamino]-4-methyl-pentanoic acid;
2-[1-carboxy-2-[3-(4-trifluoromethoxybenzyl)-3H-dazol-4-yl]ethylamino]-4-methyl-pentanoic acid;
2-[1-carboxy-2-[3-(4-isopropylbenzyl)-3H-imidazol-4-yl]ethylamino]-4-methyl-pentanoic acid;
2-[1-carboxy-2-[3-(4-tert-butylbenzyl)-3H-imidazol-4-yl]ethylamino]-4-methyl-pentanoic acid;
2-[1-carboxy-2-[3-(4-nitrobenzyl)-3H-imidazol-4-yl]ethylamino]-4-methylpentanoic acid;
2-[1-carboxy-2-[3-(2,3-dimethoxybenzyl)-3H-imidazol-4-yl]ethylamino]-4-methyl-pentanoic acid;
2-[1-carboxy-2-[3-(2,3-difluorobenzyl)-3H-imidazol-4-yl]ethylamino]-4-methyl-pentanoic acid;
2-[1-carboxy-2-[3-(2,3-dichlorobenzyl)-3H-imidazol-4-yl]ethylamino]-4-methyl-pentanoic acid;
2-[1-carboxy-2-[3-(3-trifluoromethylbenzyl)-3H-imidazol-4-yl]ethylamino]-4-methyl-pentanoic acid;
2-[2-(3-benzo[1,3]dioxol-5-ylmethyl-3H-imidazol-4-yl)-1-carboxyethylamino]-4-methylpentanoic acid;
2-[1-carboxy-2-[3-(2-cyclohexylethyl)-3H-imidazol-4-yl]ethylamino]-4-methyl-pentanoic acid;
2-[1-carboxy-2-[3-phenethyl-3H-imidazol-4-yl]ethylamino]-4-methylpentanoic acid;
2-[1-carboxy-2-[3-(3-iodobenzyl)-3H-imidazol-4-yl]ethylamino]-4-methylpentanoic acid;
2-[1-carboxy-2-[3-(3-fluorobenzyl)-3H-imidazol-4-yl]ethylamino]-4-methylpentanoic acid;
2-[1-carboxy-2-[3-benzyloxymethyl-3H-imidazol-4-yl]ethylamino]-4-methylpentanoic acid;
2-[1-carboxy-2-[3-(4-butylbenzyl)-3H-imidazol-4-yl]ethylamino]-4-methylpentanoic acid;
2-[1-carboxy-2-[3-(2-methylbenzyl)-3H-imidazol-4-yl]ethylamino]-4-methylpentanoic acid;
2-[1-carboxy-2-[2-phenylthiazol-4-yl]ethylamino]-4-methylpentanoic acid;
2-[1-carboxy-2-[1-benzyl)-1H-pyrazol-4-yl]ethylamino]-4-methylpentanoic acid; and
2-[1-carboxy-2-[3-(2-methylbiphenyl-3-ylmethyl)-3H-imidazol-4-yl]ethylamino]-4-methylpentanoic acid.

As in all embodiments, any of the above compounds can be present in the above form or in the form of a pharmaceutically acceptable salt thereof or a prodrug thereof.

The present invention is illustrated herein by particular reference to (S,S)-2-[1-carboxy-2-[3-(3,5-dichlorobenzyl)-3H-imidazol-4-yl]-ethylamino]-4-methylpentanoic acid, otherwise known as GL1001, which is the (S,S)-enantiomer of a compound having the formula

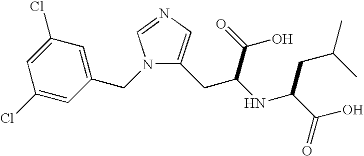

as disclosed for example by Dales et al. (2002), supra, together with a process for preparing such a compound. In brief, this process comprises treating (S)-histidine methyl ester with Boc₂O to provide a fully protected histidine derivative. The N-3 imidazole nitrogen is then selectively alkylated using the triflate of 3,5-dichlorobenzyl alcohol. Following Boc deprotection, reductive amination between the resulting alkylated histidine derivative and a β-ketoester furnishes a diester amine compound, which by hydrolysis yields 2-[1-carboxy-2-[3-(3,5-dichlorobenzyl)-3H-imidazol-4-yl]-ethylamino]-4-methylpentanoic acid as a mixture of diastereomers. The diastereomers can be separated and purified using HPLC and crystallization.

Other processes can be used to prepare GL1001, including without limitation processes described in above-referenced U.S. Pat. No. 6,632,830.

Whenever “GL1001” is mentioned herein, it will be understood that a pharmaceutically acceptable salt or prodrug of GL1001 can optionally be substituted for, or used in combination with, the free acid form of the drug, unless expressly indicated otherwise.

Additional compounds having ACE2 inhibitory activity that can be used in practice of the present invention have been disclosed by Huentelman et al. (2004), supra, including NAAE (N-(2-aminoethyl)-1-aziridineethanamine).

Further additional compounds having ACE2 inhibitory activity that can be used in practice of the present invention have been disclosed by Rella et al. (2006) J. Chem. Inf. Model. 46(2):708-716. This publication discloses structure-based pharmacophore design and virtual screening for novel ACE2 inhibitors, including 17 compounds that are reported to display an inhibitory effect on ACE2 activity, the six most active exhibiting IC₅₀ values in the range of 62-179 μM.

Methods provided herein are useful in managing and treating inflammatory, erosive, dyspeptic and/or reflux disorders of the whole or any part or parts of the proximal digestive tract of a subject. Such disorders include, without limitation:
esophagitis (acute or chronic);
acute gastritis;
chronic gastritis;
antral gastritis (acute or chronic);
pyloritis (acute or chronic);
duodenitis (acute or chronic);
H. pylori-positive gastritis;
H. pylori-positive duodenitis;
H. pylori-negative gastritis;
H. pylori-negative duodenitis;
NSAID-induced acute gastritis;
NSAID-induced chronic gastritis;
NSAID-induced duodenitis (acute or chronic);
reflux gastritis;
reflux esophagitis;
erosive esophagitis;
esophageal ulcer;
autoimmune disease-induced esophagitis;
autoimmune disease-induced gastritis;
autoimmune disease-induced duodenitis;
gastroduodenal Crohn's disease;
Zollinger-Ellison syndrome;
idiopathic inflammatory disease of esophagus, stomach and/or duodenum;
celiac sprue;
inflammatory gastroparesis;
atrophic gastritis;
pernicious anemia;
peptic ulcer disease;
gastric ulcer;
duodenal ulcer;
hemorrhagic gastritis;
hemorrhagic duodenitis;
perforated ulcer;
dyspepsia;
heartburn;
functional dyspepsia;
postprandial distress syndrome;
epigastric pain syndrome;
GERD (acute or chronic);
NERD;
acid regurgitation; and
Barrett's esophagus.

It will be understood that two or more of the above conditions can occur simultaneously, and may be part of a more general syndrome. Inflammation occurring in the esophagus and stomach, for example, may be referred to as gastroesophagitis; likewise, inflammation occurring in the stomach and duodenum may be referred to as gastroduodenitis. Gastroduodenal ulcer refers to an erosive or ulcerative condition present in both stomach and duodenum.

In certain embodiments of the invention, the disorder managed or treated is other than chronic gastritis or Crohn's disease.

The terms “disorder,” “disease” and “condition” are used interchangeably herein, unless the particular context demands that a distinction be drawn.

Unless the context demands otherwise, the terms “treat,” “treating” or “treatment” herein include preventive or prophylactic use of an agent in a subject at risk of, or having a prognosis including, an inflammatory, erosive, dyspeptic or reflux disorder of the proximal digestive tract, as well as use of such an agent in a subject already experiencing such a disorder, as a therapy to alleviate, relieve, reduce intensity of or eliminate one or more symptoms of the disorder or an underlying cause thereof. Thus treatment includes (a) preventing a disorder from occurring in a subject that may be predisposed to the disorder but in whom the disorder has not yet been diagnosed; (b) inhibiting progression of the disorder, including arresting its development; and/or (c) relieving, alleviating or ameliorating the disorder, or primary or secondary signs and symptoms thereof, including promoting, inducing or maintaining remission of the disorder. The terms “prevent,” “preventing,” “prevention” and “preventive” will be understood to have their normal meaning in the medical arts of reducing risk or future incidence or severity of a disorder, or of one or more symptoms thereof, as opposed to total elimination of future occurrence of the disorder or symptoms.

In one embodiment of the invention, a method is provided for protecting from erosion or ulceration a mucosa surface of the proximal digestive tract of a subject at risk therefor, comprising administering to the subject a therapeutically effective amount of an ACE2 inhibitor or a compound of formula

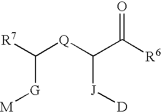

as defined above, e.g., GL1001. For example, such a method can provide protection from duodenal, gastric and/or esophageal ulcer formation, development or recurrence related to concomitantly administered medication, e.g., medication comprising an NSAID.

In another embodiment of the invention, a method is provided for promoting healing of mucosal ulceration in a subject having an inflammatory or erosive disorder of the proximal digestive tract, comprising administering to the subject a therapeutically effective amount of an ACE2 inhibitor or a compound of formula

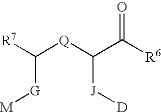

as defined above, e.g., GL1001. For example, such a method can promote healing of duodenal, gastric and/or esophageal ulcers arising from any cause including but not limited to concomitantly administered medication, e.g., comprising an NSAID.

In yet another embodiment of the invention, a method is provided for maintaining remission of mucosal ulceration in a subject having an inflammatory or erosive disorder of the proximal digestive tract, comprising administering to the subject a therapeutically effective amount of an ACE2 inhibitor or a compound of formula

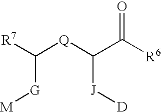

as defined above, e.g., GL1001. For example, such a method can maintain remission from duodenal, gastric and/or esophageal ulcers arising from any cause including but not limited to concomitantly administered medication, e.g., comprising an NSAID. “Maintaining remission” herein means extending or prolonging a period of remission between healing of an ulcer and re-formation of an ulcer at the same or a different locus in the proximal digestive tract.

According to any of the methods described hereinabove, the compound can be administered in monotherapy or in combination or adjunctive therapy with another agent for management or treatment of an inflammatory, erosive, dyspeptic or reflux disorder of the proximal digestive tract, including any such disorder illustratively listed above. Thus an embodiment of the invention comprises administering, in combination or adjunctive therapy, to a subject having or at risk of such a disorder (a) an ACE2 inhibitor or a compound of formula

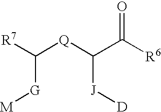

as defined above, e.g., GL1001, and (b) a second agent for management or treatment of the disorder, in therapeutically effective absolute and relative amounts. It will be recognized that what constitutes a therapeutically effective amount of a drug in combination or adjunctive therapy may differ from that in monotherapy, having regard to possible adverse interactions between drugs, co-action of drugs having different modes of action, etc.

Examples of drugs suitable as the second agent include, without limitation, PPIs, e.g., omeprazole (or its S-enantiomer esomeprazole), lansoprazole, pantoprazole or rabeprazole; H₂ receptor antagonists, e.g., cimetidine, famotidine or ranitidine; antacids, e.g., bismuth subsalicylate; prostaglandins, e.g., misoprostol; sucralfate; and, in the case of H. pylori-positive inflammatory or erosive disease, antibiotics, e.g., amoxicillin, clarithromycin, metronidazole or tetracycline. Multiple combination therapies including three or more drugs are also contemplated, for example GL 1001+rabeprazole+metronidazole+amoxicillin.

It is believed, without being bound by theory, that the mode of action of an ACE2 inhibitor or a compound of formula

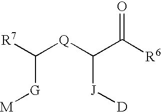

as defined above, e.g., GL1001, can be complementary to that of a PPI or H₂ receptor antagonist; and further that its efficacy is not negated by lack of efficacy of PPIs or H₂ receptor antagonists. In one embodiment, therefore, an ACE2 inhibitor or compound of formula

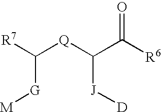

as defined above, e.g., GL1001, is administered to a subject having an inflammatory, erosive, dyspeptic or reflux disorder of the proximal digestive tract that is refractory to PPI or H₂ receptor antagonist treatment. Such administration can be in monotherapy or in combination or adjunctive therapy as described above.

A “subject” herein is a warm-blooded animal, generally a mammal such as, for example, a cat, dog, horse, cow, pig, mouse, rat or primate, including a human. In one embodiment the subject is human, for example a patient having a clinically diagnosed inflammatory, erosive, dyspeptic or reflux disorder of the proximal digestive tract such as any of those illustratively listed above. Animal models in experimental investigations relevant to human disease are also examples of “subjects” herein, and can include for example rodents (e.g., mouse, rat, guinea pig), lagomorphs (e.g., rabbit), carnivores (e.g., cat, dog), or nonhuman primates (e.g., monkey, chimpanzee). Further, the subject can be an animal (for example a domestic, farm, working, sporting or zoo animal) in veterinary care.

Certain compounds useful according to the present invention have acid and/or base moieties that, under suitable conditions, can form salts with suitable acids. For example, GL1001 has two acid moieties that, under suitable conditions, can form salts with suitable bases, and an amino group that, under suitable conditions, can form salts with suitable acids. Internal salts can also be formed. The compound can be used in its free acid/base form or in the form of an internal salt, an acid addition salt or a salt with a base.

Acid addition salts can illustratively be formed with inorganic acids such as mineral acids, for example sulfuric acid, phosphoric acids or hydrohalic (e.g., hydrochloric or hydrobromic) acids; with organic carboxylic acids such as (a) C₁₋₄ alkanecarboxylic acids which may be unsubstituted or substituted (e.g., halosubstituted), for example acetic acid, (b) saturated or unsaturated dicarboxylic acids, for example oxalic, malonic, succinic, maleic, fumaric, phthalic or terephthalic acids, (c) hydroxycarboxylic acids, for example ascorbic, glycolic, lactic, malic, tartaric or citric acids, (d) amino acids, for example aspartic or glutamic acids, or (e) benzoic acid; or with organic sulfonic acids such as C₁₋₄ alkanesulfonic acids or arylsulfonic acids which may be unsubstituted (e.g., halosubstituted), for example methanesulfonic acid or p-toluenesulfonic acid.

Salts with bases include metal salts such as alkali metal or alkaline earth metal salts, for example sodium, potassium or magnesium salts; or salts with ammonia or an organic amine such as morpholine, thiomorpholine, piperidine, pyrrolidine, a mono-, di- or tri-lower alkyl amine, for example ethylamine, tert-butylamine, diethylamine, diisopropylamine, triethylamine, tributylamine or dimethylpropylamine, or a mono-, di- or tri-(hydroxy lower alkyl) amine, for example monoethanolamine, diethanolamine or triethanolamine.

Alternatively, a prodrug of the compound or a salt of such prodrug can be used. A prodrug is a compound, typically itself having weak or no pharmaceutical activity, that is cleaved, metabolized or otherwise converted in the body of a subject to an active compound. Examples of prodrugs are esters, particularly alkanoyl esters and more particularly C₁₋₆ alkanoyl esters. Other examples include carbamates, carbonates, ketals, acetals, phosphates, phosphonates, sulfates and sulfonates. Various prodrugs of GL1001, and methods of making such prodrugs, are disclosed, for instance, in above-referenced U.S. Pat. No. 6,632,830 and U.S. Published Patent Application No. 2004/0082496.

According to any embodiment of the invention herein that specifies a compound or a salt thereof or a prodrug thereof, in one aspect of such embodiment the agent in question is used in the form of the compound (as free base or free acid) or a pharmaceutically acceptable salt thereof.

The compound should be administered in a therapeutically effective amount. What constitutes a therapeutically effective amount depends on a number of factors, including the particular subject's age and body weight, the nature, stage and severity of the disease, the particular effect sought (e.g., reduction of inflammation, alleviation of symptoms, healing of ulcers, maintenance of remission, etc.) and other factors, but for most subjects a dosage amount of about 0.5 to about 5000 mg/day, more typically about 5 to about 2000 mg/day, will be found suitable. In particular embodiments, the dosage employed is about 10 to about 1800 mg/day, about 50 to about 1600 mg/day or about 100 to about 1500 mg/day; illustratively about 50, about 100, about 150, about 200, about 250, about 300, about 350, about 400, about 450, about 500, about 550, about 600, about 650, about 700, about 750, about 800, about 900, about 1000, about 1100, about 1200, about 1300, about 1400, about 1500 or about 1600 mg/day.

Where a salt or prodrug of the compound is used, the amount administered should be an amount delivering a daily dosage of the compound as set forth above.

Thus in one embodiment a method of the invention comprises administering to the subject an ACE2 inhibitor or a compound of formula

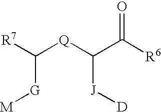

as defined above, e.g., GL1001, in an amount of about 0.5 to about 5000 mg/day, for example about 5 to about 2000 mg/day, about 10 to about 1800 mg/day, about 50 to about 1600 mg/day or about 100 to about 1500 mg/day; illustratively about 50, about 100, about 150, about 200, about 250, about 300, about 350, about 400, about 450, about 500, about 550, about 600, about 650, about 700, about 750, about 800, about 900, about 1000, about 1100, about 1200, about 1300, about 1400, about 1500 or about 1600 mg/day.

The above dosages are given on a per diem basis but should not be interpreted as necessarily being administered on a once daily frequency. Indeed the compound, or salt or prodrug thereof, can be administered at any suitable frequency, for example as determined conventionally by a physician taking into account a number of factors, but typically about four times a day, three times a day, twice a day, once a day, every second day, twice a week, once a week, ice a month or once a month. The compound, or salt or prodrug thereof, can alternatively be administered more or less continuously, for example by parenteral infusion in a hospital setting. In some situations a single dose may be administered, but more typically administration is according to a regimen involving repeated dosage over a treatment period. In such a regimen the daily dosage and/or frequency of administration can, if desired, be varied over the course of the treatment period, for example introducing the subject to the compound at a relatively low dose and then increasing the dose in one or more steps until a full dose is reached.

The treatment period is generally as long as is needed to achieve a desired outcome, for example induction or maintenance of remission, alleviation of symptoms, healing of ulcers, etc. In some situations it will be found useful to administer the drug intermittently, for example for treatment periods of days, weeks or months separated by non-treatment periods. Such intermittent administration can be timed, for example, to correspond to flares of the disorder.

Administration can be by any suitable route, including without limitation oral, buccal, sublingual, intranasal, intraocular, rectal, vaginal, transdermal or parenteral (e.g., intradermal, subcutaneous, intramuscular, intravenous, intra-arterial, intratracheal, intraventricular, intraperitoneal, etc.) routes, and including by inhalation or implantation.

While it can be possible to administer the compound, or a salt or prodrug thereof unformulated as active pharmaceutical ingredient (API) alone, it will generally be found preferable to administer the API in a pharmaceutical composition that comprises the API and at least one pharmaceutically acceptable excipient. The excipient(s) collectively provide a vehicle or carrier for the API. Pharmaceutical compositions adapted for all possible routes of administration are well known in the art and can be prepared according to principles and procedures set forth in standard texts and handbooks such as those individually cited below.

USIP, ed. (2005) Remington: The Science and Practice of Pharmacy, 21st ed., Lippincott, Williams & Wilkins.

Allen et al. (2004) Ansel's Pharmaceutical Dosage Forms and Drug Delivery Systems, 8th ed., Lippincott, Williams & Wilkins.

Suitable excipients are described, for example, in Kibbe, ed. (2000) Handbook of Pharmaceutical Excipients, 3rd ed., American Pharmaceutical Association.

Examples of formulations that can be used as vehicles for delivery of the API in practice of the present invention include, without limitation, solutions, suspensions, powders, granules, tablets, capsules, pills, lozenges, chews, creams, ointments, gels, liposomal preparations, nanoparticulate preparations, injectable preparations, enemas, suppositories, inhalable powders, sprayable liquids, aerosols, patches, depots and implants. Illustratively, in a liquid formulation suitable, for example, for parenteral, intranasal or oral delivery, the API can be present in solution or suspension, or in some other form of dispersion, in a liquid medium that comprises a diluent such as water. Additional excipients that can be present in such a formulation include a tonicifying agent, a buffer (e.g., a tris, phosphate, imidazole or bicarbonate buffer), a dispersing or suspending agent and/or a preservative. Such a formulation can contain micro- or nanoparticulates, micelles and/or liposomes. A parenteral formulation can be prepared in dry reconstitutable form, requiring addition of a liquid carrier such as water or saline prior to administration by injection.

For rectal delivery, the API can be present in dispersed form in a suitable liquid (e.g., as an enema), semi-solid (e.g., as a cream or ointment) or solid (e.g., as a suppository) medium. The medium can be hydrophilic or lipophilic.

For oral delivery, the API can be formulated in liquid or solid form, for example as a solid unit dosage form such as a tablet or capsule. Such a dosage form typically comprises as excipients one or more pharmaceutically acceptable diluents, binding agents, disintegrants, wetting agents and/or antifrictional agents (lubricants, anti-adherents and/or glidants). Many excipients have two or more functions in a pharmaceutical composition. Characterization herein of a particular excipient as having a certain function, e.g., diluent, binding agent, disintegrant, etc., should not be read as limiting to that function.

Suitable diluents illustratively include, either individually or in combination, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches (e.g., corn starch, wheat starch, rice starch, potato starch, tapioca starch, etc.), starch components such as amylose and dextrates, and modified or processed starches such as pregelattized starch; dextrins; celluloses including powdered cellulose, microcrystalline cellulose, silicified microcrystalline cellulose, food grade sources of α- and amorphous cellulose and powdered cellulose, and cellulose acetate; calcium salts including calcium carbonate, tribasic calcium phosphate, dibasic calcium phosphate dihydrate, monobasic calcium sulfate monohydrate, calcium sulfate and granular calcium lactate trihydrate; magnesium carbonate; magnesium oxide; bentonite; kaolin; sodium chloride; and the like. Such diluents, if present, typically constitute in total about 5% to about 99%, for example about 10% to about 85%, or about 20% to about 80%, by weight of the composition. The diluent or diluents selected preferably exhibit suitable flow properties and, where tablets are desired, compressibility.

Lactose, microcrystalline cellulose and starch, either individually or in combination, are particularly useful diluents.

Binding agents or adhesives are useful excipients, particularly where the composition is in the form of a tablet. Such binding agents and adhesives should impart sufficient cohesion to the blend being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; glucose; polydextrose; starch including pregelatinized starch; gelatin; modified celluloses including methylcellulose, carmellose sodium, hydroxypropylmethylcellulose (HPMC or bypromellose), hydroxypropyl-cellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone), for example povidone K-15, K-30 and K-29/32; polyacrylic acids (carbomers); polymethacrylates; and the like. One or more binding agents and/or adhesives, if present, typically constitute in total about 0.5% to about 25%, for example about 0.75% to about 15%, or about 1% to about 10%, by weight of the composition.

Povidone is a particularly useful binding agent for tablet formulations, and, if present, typically constitutes about 0.5% to about 15%, for example about 1% to about 10%, or about 2% to about 8%, by weight of the composition.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcelluose, low-substituted hydroxypropylcellulose, carmellose, carmellose calcium, carmellose sodium and croscarmellose sodium; alginates; povidone; crospovidone; polacrilin potassium; gums such as agar, gpar, locust bean, karaya, pectin and tragacanth gums; colloidal silicon dioxide; and the like. One or more disintegrants, if present, typically constitute in total about 0.2% to about 30%, for example about 0.2% to about 10%, or about 0.2% to about 5%, by weight of the composition.

Croscarmellose sodium and crospovidone, either individually or in combination, are particularly useful disintegrants for tablet or capsule formulations, and, if present, typically constitute in total about 0.2% to about 10%, for example about 0.5% to about 7%, or about 1% to about 5%, by weight of the composition.

Wetting agents, if present, are normally selected to maintain the drug or drugs in close association with water, a condition that is believed to improve bioavailability of the composition. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters; polyoxyethylene sorbitan esters, for example polysorbate 20 and polysorbate 80; propylene glycol fatty acid esters, for example propylene glycol laurate; sodium lauryl sulfate; fatty acids and salts thereof for example oleic acid, sodium oleate and triethanolamine oleate; glyceryl fatty acid esters, for example glyceryl monooleate, glyceryl monostearate and glyceryl palmitostearate; sorbitan esters, for example sorbitan monolaurate, sorbitan monooleate, sorbitan monopalmitate and sorbitan monostearate; tyloxapol; and the like. One or more wetting agents, if present, typically constitute in total about 0.25% to about 15%, preferably about 0.4% to about 10%, and more preferably about 0.5% to about 5%, by weight of the composition.

Wetting agents that are anionic surfactants are particularly useful. Illustratively, sodium lauryl sulfate, if present, typically constitutes about 0.25% to about 7%, for example about 0.4% to about 4%, or about 0.5% to about 2%, by weight of the composition.

Lubricants reduce friction between a tableting mixture and tableting equipment during compression of tablet formulations. Suitable lubricants include, either individually or in combination, glyceryl behenate; stearic acid and salts thereof including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. One or more lubricants, if present, typically constitute in total about 0.05% to about 10%, for example about 0.1% to about 8%, or about 0.2% to about 5%, by weight of the composition. Magnesium stearate is a particularly useful lubricant.

Anti-adherents reduce sticking of a tablet formulation to equipment surfaces. Suitable anti-adherents include, either individually or in combination, talc, colloidal silicon dioxide, starch, DL-leucine, sodium lauryl sulfate and metallic stearates. One or more anti-adherents, if present, typically constitute in total about 0.1% to about 10%, for example about 0.1% to about 5%, or about 0.1% to about 2%, by weight of the composition.

Glidants improve flow properties and reduce static in a tableting mixture. Suitable glidants include, either individually or in combination, colloidal silicon dioxide, starch, powdered cellulose, sodium lauryl sulfate, magnesium trisilicate and metallic stearates. One or more glidants, if present, typically constitute in total about 0.1% to about 10%, for example about 0.1% to about 5%, or about 0.1% to about 2%, by weight of the composition.

Talc and colloidal silicon dioxide, either individually or in combination, are particularly useful anti-adherents and glidants.

Other excipients such as buffering agents, stabilizers, antioxidants, antimicrobials, colorants, flavors and sweeteners are known in the pharmaceutical art and can be used. Tablets can be uncoated or can comprise a core that is coated, for example with a nonfunctional film or a release-modifying or enteric coating. Capsules can have hard or soft shells comprising, for example, gelatin and/or HPMC, optionally together with one or more plasticizers.

A pharmaceutical composition useful herein typically contains the compound or salt or prodrug thereof in an amount of about 1% to about 99%, more typically about 5% to about 90% or about 10% to about 60%, by weight of the composition. A unit dosage form such as a tablet or capsule can conveniently contain an amount of the compound providing a single dose, although where the dose required is large it may be necessary or desirable to administer a plurality of dosage forms as a single dose. Illustratively, a unit dosage form can comprise the compound in an amount of about 10 to about 1800 mg, for example about 50 to about 1600 mg or about 100 to about 1500 mg; illustratively about 50, about 100, about 150, about 200, about 250, about 300, about 350, about 400, about 450, about 500, about 550, about 600, about 650, about 700, about 750, about 800, about 900, about 1000, about 1100, about 1200, about 1300, about 1400, about 1500 or about 1600 mg.

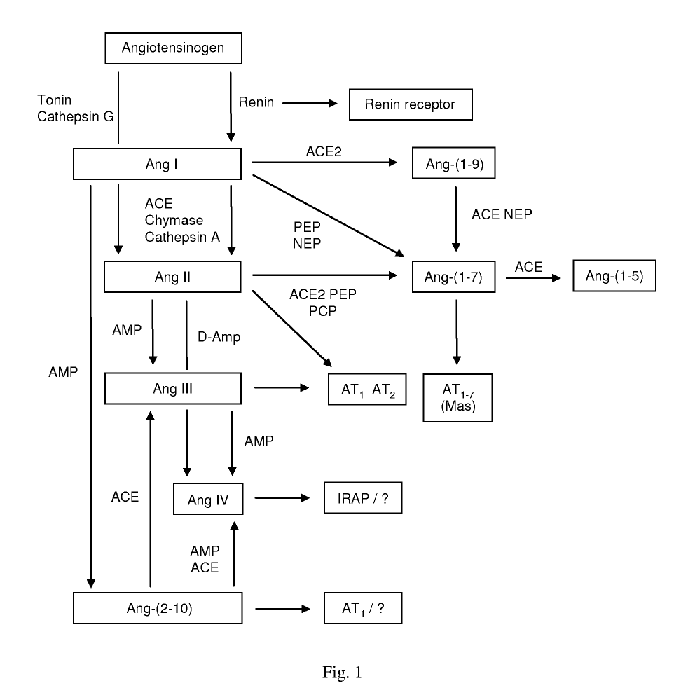

The effect of an ACE2 inhibitor on the renin-angiotensin system (RAS) might be predicted to involve increase in level of Ang II (see FIG. 1), which, as indicated above is implicated in a variety of pro-inflammatory effects. The present inventors have found, contrary to such prediction, that activation of NF-κB, a key mediator for synthesis of pro-inflammatory cytokines, is not promoted but inhibited by the ACE2 inhibitor.

For example, it has surprisingly been found that the ACE2 inhibitor GL1001 inhibits in vivo basal NF-κB dependent transcription in recombinant reporter mice. This finding is reported in greater detail in Example 2 below, and appears to support an anti-inflammatory effect of the ACE2 inhibitor that is contrary to expectation based on present understanding of the role of ACE2 in the RAS.

It has still further surprisingly been found that ACE2 mRNA expression in tissues of the digestive tract is especially strongly elevated in chronic gastritis. It is accordingly contemplated that elevation of ACE2 in chronic gastritis is a potential pathogenic factor in that disease and that administration of an ACE2 inhibitor such as GL100 is beneficial in treatment of chronic gastritis. Confirmation of the effectiveness of GL1001 in a chronic gastritis model is seen in Example 4 below. This finding is especially surprising in view of studies reported in the literature (e.g., Fleichman et a. (2002), supra) showing that Ang 11 (the level of which would be expected to increase following treatment with an ACE2 inhibitor) is associated with increased gastric mucosal proliferation and that lowering of Ang II, for example with the ACE inhibitor enalapril, promotes healing of gastric mucosal erosions.

It is further contemplated that other inflammatory, erosive, dyspeptic and reflux disorders of the proximal digestive tract including acute gastritis, acute or chronic duodenitis, acute or chronic esophagitis, duodenal ulcer, celiac sprue, functional dyspepsia and GERD, can be similarly responsive to an ACE2 inhibitor such as GL1001. In Examples 5 and 6 below, GL1101 is shown to provide gastroprotection from acute NSAID toxicity. The term “gastroprotection” will be understood herein to extend to protection not only of the stomach but also of other parts of the digestive tract including the duodenum from NSAID-induced mucosal injury.

It is still further contemplated that where a disorder, especially an inflammatory or erosive disorder, extends to more distal parts of the digestive tract, as in the case, for example, of celiac sprue, or NSAID-induced injury to the intestinal tract (NSAID enteropathy), an ACE2 inhibitor or a compound of formula

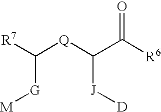

as defined above, e.g., GL1001, can have therapeutic benefit likewise extending to such more distal parts.

The term “therapeutic combination” herein refers to a plurality of agents that when administered to a subject together or separately, are co-active in bringing therapeutic benefit to the subject. Such administration is referred to as “combination therapy,” “co-therapy,” “adjunctive therapy” or “add-on therapy.” For example, one agent can potentiate or enhance the therapeutic effect of another, or reduce an adverse side effect of another, or one or more agents can be effectively administered at a lower dose than when used alone, or can provide greater therapeutic benefit than when used alone, or can complementarily address different aspects, symptoms or etiological factors of a disease or condition.

For example, a therapeutic combination is provided comprising an ACE2 inhibitor or a compound of formula

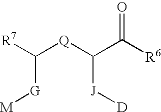

as defined above, e.g., GL1001, and at least one additional agent selected from PPIs, H2 receptor antagonists, antacids, sucralfate, prostaglandins and antibiotics. As described above, such a combination can be useful to treat an inflammatory, erosive, dyspeptic or reflux disorder of the proximal digestive tract.

In another embodiment, a therapeutic combination is provided comprising an NSAID and a gastroprotective agent that comprises an ACE2 inhibitor or a compound of formula

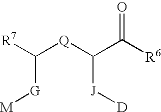

as defined above, e.g., GL1001. Such a combination can be useful to treat any disease or condition in which an NSAID is indicated, for example inflammatory diseases such as rheumatoid arthritis and osteoarthritis, musculoskeletal pain, etc. The NSAID is present in an anti-inflammatory, analgesic or antipyretic effective amount, and the gastroprotective agent is present in an amount effective to protect mucosal surfaces of the proximal digestive tract from erosion or ulceration induced by the NSAID.

Nonlimiting examples of NSAIDs useful in such a combination include salicylic acid derivatives (such as salicylic acid, acetylsalicylic acid, methyl salicylate, diflunisal, olsalazine, salsalate and sulfasalazine), indole and indene acetic acids (such as indomethacin, etodolac and sulindac), fenamates (such as etofenamic, meclofenamic, mefenamic, flufenamic, niflumic and tolfenamic acids), heteroaryl acetic acids (such as acemetacin, alelofenac, clidanac, diclofenac, fenchlofenac, fentiazac, furofenac, ibufenac, isoxepac, ketorolac, oxipinac, tiopinac, tolmetin, zidometacin and zomepirac), aryl acetic acid and propionic acid derivatives (such as alminoprofen, benoxaprofen, bucloxic acid, carprofen, fenbufen, fenoprofen, fluprofen, flurbiprofen, ibuprofen, indoprofen, ketoprofen, miroprofen, naproxen, naproxen sodium, oxaprozin, pirprofen, pranoprofen, suprofen, tiaprofenic acid and tioxaprofen), enolic acids (such as the oxicam derivatives ampiroxicam, cinnoxicam, droxicam, lomoxicam, meloxicam, piroxicam, sudoxicam and tenoxicam, and the pyrazolone derivatives aminopyrine, antipyrine, apazone, dipyrone, oxyphenbutazone and phenylbutazone), alkanones (such as nabumetone), nimesulide, proquazone, MX-1094, licofelone, pharmaceutically acceptable salts thereof, and combinations thereof

Also considered as NSAIDs are COX-2 selective inhibitors. These typically are less damaging to the proximal digestive tract mucosa than nonselective NSAIDs, but may still benefit from gastroprotection according to the present invention. Nonlimiting examples of COX-2 selective inhibitors include celecoxib, deracoxib, valdecoxib, parecoxib, rofecoxib, etoricoxib, luntracoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid, 2-(3,4-difluoro-phenyl)-4-(3-hydroxy-3-methyl-1-butoxy)-5-[4-(methylsulfonyl)phenyl]-3-(2H)-pyridazinone, 4-[5-(4-fluorophenyl)-3-(trifluoromethyl)-1H-pyrazol-1-yl]benzenesulfonamide, 4-[5-(phenyl)-3-(trifluoromethyl)-1H-pyrazol-1-yl]benzenesulfonamide, PAC-10549 cimicoxib, GW-406381, LAS-34475, CS-502, pharmaceutically acceptable salts thereof, and combinations thereof.

In specific examples of the present embodiment, the therapeutic combination comprises GL1001 and at least one NSAID selected from acetylsalicylic acid (aspirin), indomethacin, diclofenac, ibuprofen, naproxen, oxaprozin, meloxicam and celecoxib.

The two or more active agents administered in combination or adjunctive therapy can be formulated in one pharmaceutical preparation (single dosage form) for administration to the subject at the same time, or in two or more distinct preparations (separate dosage forms) for administration to the subject at the same or different times, e.g., sequentially. The two distinct preparations can be formulated for administration by the same route or by different routes.

Separate dosage forms can optionally be co-packaged, for example in a single container or in a plurality of containers within a single outer package, or co-presented in separate packaging (“common presentation”). As an example of co-packaging or common presentation, a kit is contemplated comprising, in a first container, a first agent that comprises an ACE2 inhibitor or a compound of formula

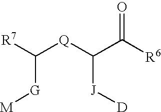

as defined above, e.g., GL1001, and, in a second container, a second agent such as any of those mentioned above. In another example, the first and second agents are separately packaged and available for sale independently of one another, but are co-marketed or co-promoted for use according to the invention. The separate dosage forms may also be presented to a subject separately and independently, for use according to the invention.

Depending on the dosage forms, which may be identical or different, e.g., fast release dosage forms, controlled release dosage forms or depot forms, the first and second agents may be administered on the same or on different schedules, for example on a daily, weekly or monthly basis.

### EXAMPLES

### Example 1

### ACE2 mRNA Expression in Normal and Disease States

Donoghue et al. (2000), supra, reported finding ACE2 transcripts mainly in heart, kidney and testis, out of 23 normal human tissues examined, and ACE2 protein (via immunohistochemistry) predominantly in the endothelium of coronary and intrarenal vessels and in renal tubular epithelium.

Further, Tipnis et al. (2000) J. Biol. Chem. 275(43):33238-33243 reported northern blotting analyses showing that the ACE2 mRNA transcript is most highly expressed in testis, kidney and heart.

Komatsu et al. (2002) DNA Seq. 13; 217-220 reported molecular cloning of mouse angiotensin-converting enzyme-related carboxypeptidase (mACE2) showing 83% identity with human ACE2, and northern blot analysis showing transcripts were expressed mainly in kidney and lungs.

More recently, Gembardt et al. (2005) Peptides 26:1270-1277 analyzed ACE2 mRNA and protein expression in various normal tissues of mice and rats, reporting at least detectable levels of ACE2 mRNA in all tested organs of both species (ventricle, kidney, lung, liver, testis, gallbladder, forebrain, spleen, thymus, stomach, ileum, colon, brainstem, atrium, and adipose tissue). In both species ileum tissue showed the highest expression of ACE2 mRNA, with the mouse exceeding the rat in ACE2 mRNA expression in this organ and also in the kidney and colon.

Burrel et al. (2005) Eur. Heart J. 26:369-375 recently reported that myocardial infarction increases ACE2 expression in rat and human heart.

ACE2 mRNA expression has now been examined in various human tissues from normal and diseased subjects, using the BioExpress® System. T is system includes mRNA expression data from about 18,000 samples, of which about 90% are from human tissues, comprising both normal and diseased samples from about 435 disease states. In brief, human tissue samples, either from surgical biopsy or post-mortem removal, were processed for mRNA expression profile analysis using Affymetrix GeneChips®. Each tissue sample was examined by a board-certified pathologist to confirm pathological diagnoses. RNA isolation, cDNA synthesis, cRNA amplification and labeling, hybridizations, and signal normalization were carried out using standard Affymetrix protocols. Computational analysis was performed using Genesis Enterprise System® Software and the Ascenta® software system.

In agreement with Donoghue et al. (2000), supra, and Tipnis et al. (2000), supra, the present study showed relatively high levels of ACE2 transcripts in normal human heart, kidney and testis (data not shown). However, excluding those three normal tissues, the top 8 highest expression levels of ACE2 mRNA in 70 additional normal human tissues that were examined are listed in Table 1 below, in descending order of mean expression level (given as the “average relative level,” i.e., sample set signal level in arbitrary units, normalized to the lowest signal level in all tested samples, averaged for two different probe fragments).

These top 8 normal tissues in Table 1 (and heart, kidney and testis as well) showed average relative levels of ACE2 mRNA expression greater than 4.0, while the remaining 62 normal tissues examined showed average relative levels less than 4.0.

Table 1 also shows that four of the top five highest expression levels of ACE2 mRNA in normal human tissues (other than heart, kidney and testis) were in components of the gastrointestinal tract, namely (in descending order of expression level): duodenum, small intestine, colon and stomach.

TABLE 1
Relative levels of ACE2 mRNA expression in normal tissues

| | Average Relative |
|---|---|
| Sample Set | Level |
| Duodenum | 221.2 |
| Small Intestine | 167.9 |
| Gallbladder | 109.9 |
| Colon | 13.6 |
| Stomach | 10.1 |
| Ovary | 5.7 |
| Pancreas | 4.3 |
| Liver | 4.2 |

Examination of ACE2 mRNA expression in disease states encompassed by the BioExpress® System showed elevation of ACE2 mRNA in only a few conditions, mainly inflammatory conditions of components of the gastrointestinal tract. Thus, Table 2 shows that ACE2 mRNA expression was elevated (in descending order of average fold change vs. normal) in inflammatory conditions of the stomach (chronic gastritis), major salivary gland (excluding parotid) (chronic sialadenitis), and colon (Crohn's disease, active (chronic or acute inflammation)). In contrast, the levels of ACE2 mRNA in colon with active ulcerative colitis (chronic or acute inflammation), and in small intestine with active Crohn's disease (chronic or acute inflammation), were substantially unchanged from the already significant levels in corresponding normal tissues shown in Table 1.

TABLE 2
Effects of inflammatory conditions on ACE2
mRNA expression in digestive tract tissues

| | Average Fold |
|---|---|
| | Change vs. |
| Sample Set | Normal |
| Stomach, chronic gastritis | 8.2 |
| Major salivary gland (excluding parotid), chronic | 7.5 |
| sialadenitis | |
| Colon, Crohn's disease, active (chronic inflammation) | 2.2 |
| Colon, Crohn's disease, active (acute inflammation) | 1.7 |
| Colon, ulcerative colitis, active (chronic inflammation) | 0.9 |
| Colon, ulcerative colitis, active (acute inflammation) | 1.0 |
| Small intestine, Crohn's disease, active (chronic | 0.4 |
| inflammation) | |
| Small Intestine, Crohn's disease, active (acute | 0.8 |
| inflammation) | |

The above findings taken together show that 4 of the top 11 highest expression levels of ACE2 mRNA found in normal human tissues are in components of the digestive tract, and that the majority of examined disease conditions that involve elevated ACE2 mRNA expression are inflammatory conditions of the digestive tract. Accordingly, these findings suggest that high levels of ACE2 mRNA expression could be a pathogenic factor and, hence, reduction of ACE2 activity could provide therapeutic benefit, in at least some inflammatory conditions of the digestive tract, particularly in the stomach (chronic gastritis), major salivary gland (chronic sialadenitis), and colon (Crohn's disease with chronic or acute inflammation). Further, although ACE2 mRNA levels were not elevated in colon with ulcerative colitis or small intestine with Crohn's disease, the already substantial levels of such mRNA in normal colon and small intestine suggest at least that ACE2 activity is present and, therefore, could still constitute a pathogenic factor in these two diseased tissues.

### Example 2

### Inhibition by GL1001 of In Vivo Basal NF-κB Dependent Transcription in Recombinant Reporter Mice

At least in inflammatory bowel disease, inflammation is likely to depend, at least in part, on activation and nuclear translocation of NF-dB family members. See, e.g., Fichtner-Feigl et al. (2005) J. Cin. Invest. 115:3057-3071 and sources cited therein. Thus, in Th1-mediated inflammations dependent on IL-12 and/or IL-23, synthesis of these cytokines is regulated by NF-κB transcription factors. In Th2-mediated inflammations dependent on IL-4 or IL-13, synthesis of these cytokines is also dependent on NF-κB transcription factors, albeit more indirectly than that of IL-12 and IL-23. Thus one method of treating the inflammation can be to administer agents that inhibit NF-κB activity, and indeed Fichtner-Feigl et al. (2005), supra, have shown that NF-κB decoy oligodeoxynucleotides (ODNs) that prevent NF-κB activation of gene expression are effective in treating and preventing various models of Th1- and Th2-mediated inflammatory bowel disease in mice, including acute trinitrobenzene sulfonic acid (TNBS) induced colitis, as assessed by clinical course and effect on Th1 cytokine production; chronic TNBS induced colitis, inhibiting both production of IL-23/IL-17 and development of fibrosis; and oxazolone-induced colitis, a Th2-mediated inflammatory process.

GL1001 was tested for in vivo anti-inflammatory activity by examining its effects on basal levels of NF-κB dependent transcription in mice engineered in the germline with a construct containing an NF-κB enhancer linked to a luciferase gene (i.e., NF-κB::Luc mice), such that this NE-κB reporter construct is present in all cells of the mice.

More particularly, transgenic NF-κB::Luc mice were generated using three NF-κB response elements from the Igκ light chain promoter fused to a firefly luciferase gene as described by Carlsen et al. (2002) J. Immunol. 168:1441-1446. Pronuclear microinjection of purified construct DNA was used to generate transgenic founders in the C57BL/6 XCBA/J background. Founders were subsequently back-crossed to the C57BL/6 albino background. All experimental protocols were approved by the Institutional Animal Care and Use Committee and conform to the ILAR guide for the care and used of laboratory animals. For in vivo imaging, NF-κB::Luc mice were injected intraperitoneally with luciferin (150 mg/1 kg) 10 minutes before imaging, anesthetized (using 1-3% isoflurane) and placed into a light-tight camera box. Mice were imaged for up to two minutes from the dorsal or ventral aspects at high-resolution settings with a field of view of 20 cm. The light emitted from the transgene was detected by an IVIS® Imaging System 200 Series (Xenogen Corporation, Alameda, Calif.), digitized and displayed on a monitor. The Living Images software (Xenogen Corporation, Alameda, Calif.; see Rice et al. (2002) J. Biomed. Opt. 6:432×40) displays data from the camera using a pseudocolor scale with colors representing variations of signal intensity. Signal data were also quantitated and archived using the Living Image® software. Photons of light were quantitated using an oval region of interest (ROI) of varying sizes depending on the procedure, as described further below.

For luciferase assays, organs were extracted and snap-frozen in liquid nitrogen. All tissue samples were placed in lysis buffer with inhibitors (Passive Lysis Buffer (Promega) and Complete Mini Protease Inhibitor Cocktail (Roche, Indianapolis, Ind.)), and were homogenized using a Handishear hand-held tissue homogenizer (VirTis, Gardiner, N.Y.). Tissue homogenates were centrifuged and clarified lysates were used for luminometer assays and western blots. For the luminometer assays, Luciferase Assay Substrate (Luciferase Assay System, Promega) was prepared as indicated by the manufacturer and placed in disposable cuvettes. Tissue homogenates (20 μl) and substrate (100 μl) were mixed and measurements were taken in a Veritas Microplate Luminometer (Turner Designs, Sunnyvale, Calif.) with the parameters of a 2-second delay, 10-second. Background luminescence readings were obtained and the background readings were subtracted from the luminescent data. Protein concentrations were determined using the BCA Protein Assay Kit (Pierce, Rockford, Ill.) following the manufacturer's protocols and analyzed using a VERSAmax tunable microplate reader and associated Softmax Pro version 3.1.2 software (Molecular Devices, Sunnyvale Calif.). The luminescence for each of the protein lysates was calculated as arbitrary units of light per microgram of protein. Statistical analyses include MEAN, SEM and ANOVA and Student's t-test between treatment groups.

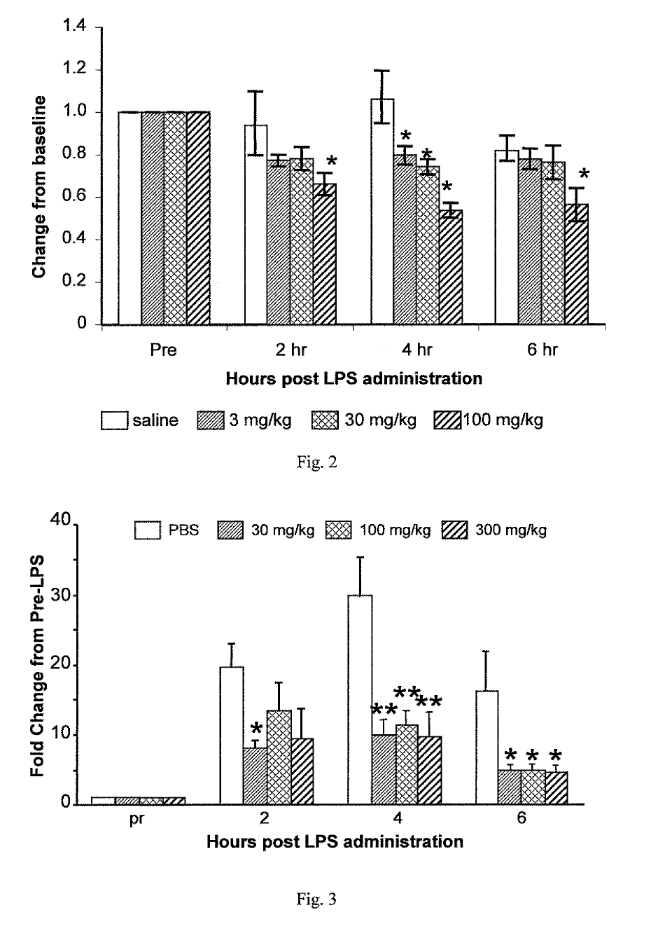

To test for in vivo effects of GL1001 on basal levels of NF-κB dependent transcription, male NF-κB::Luc mice were subjected to quantitative in vivo imaging of the abdominal area (using a fixed ROT of 2.76×3.7 cm) as described above, immediately before, and at 2, 4 and 6 hours after subcutaneous administration of 0, 3, 30 or 100 mg/kg GL1001 in saline. Whole body imaging showed that GL1001 significantly inhibited basal in vivo levels of NF-κB dependent transcription of the luciferase reporter gene, primarily in the abdominal region. As shown by the quantitative imaging data in FIG. 2, at 4 hours post-LPS administration GL1001 significantly inhibited basal in vivo levels of NF-κB dependent transcription in the selected abdominal ROT by over 40% at 300 mg/kg (p<0.01 by ANOVA and Student's t-test), and to lesser but still significant extents at both lower doses.

In contrast to the results observed in NF-κB::Luc mice, no significant effect of GL1001 was observed on basal in vivo levels of reporter luciferase expression in AP-1::Luc mice constructed similarly to the present NF-κB::Luc mice (data not shown), in which reporter transcription was driven by an enhancer element responsive to activator protein-1 (AP-1), a known proto-oncogene thought to be involved in cell proliferation and tumor promotion.

### Example 3

### GL1001 inhibits in vivo LPS-induced NF-κB Dependent Transcription in Recombinant Reporter Mice

Bacterial lipopolysaccharide (LPS), a major component of the cell wall of gram-negative bacteria, is a highly biologically active molecule which stimulates macrophages to produce and release TNFα. See, e.g., Jersmann et al. (2001) Infection and Immunity 69(3):1273-1279, and sources cited therein. One of the recognized associations of bacterial infection with cardiovascular events is activation of endothelium and up-regulation of adhesion molecules. The two major proinflammatory mediators implicated in the causation of cardiovascular events, bacterial LPS and TNFα, have been found to cooperate to enhance the adhesive properties of endothelial cells by synergistically increasing expression of human endothelial adhesion molecules through activation of NF-κB and p38 mitogen-activated protein kinase signaling pathways.

GL1001 was further tested for in vivo anti-inflammatory activity by examining its effects on bacterial LPS-induced NF-κB dependent transcription in NF-κB::Luc mice. In particular, inflammation was induced in these mice at 6-10 weeks of age by administration of 0.5 mg/kg (i.v.) soluble LPS (sLPS; Sigma) one hour after administration of GL1001. Mice were subjected to quantitative abdominal imaging at 2, 4 and 6 hours following LPS administration, as described above. In confirmatory experiments, and at the time point with the greatest modulation of luciferase signal, animals were euthanized and tissues were collected and preserved for further analysis. Luciferase signal was quantitated from several regions of interest. Statistical analyses include MEAN, SEM and ANOVA and Student's t-test between treatment groups.

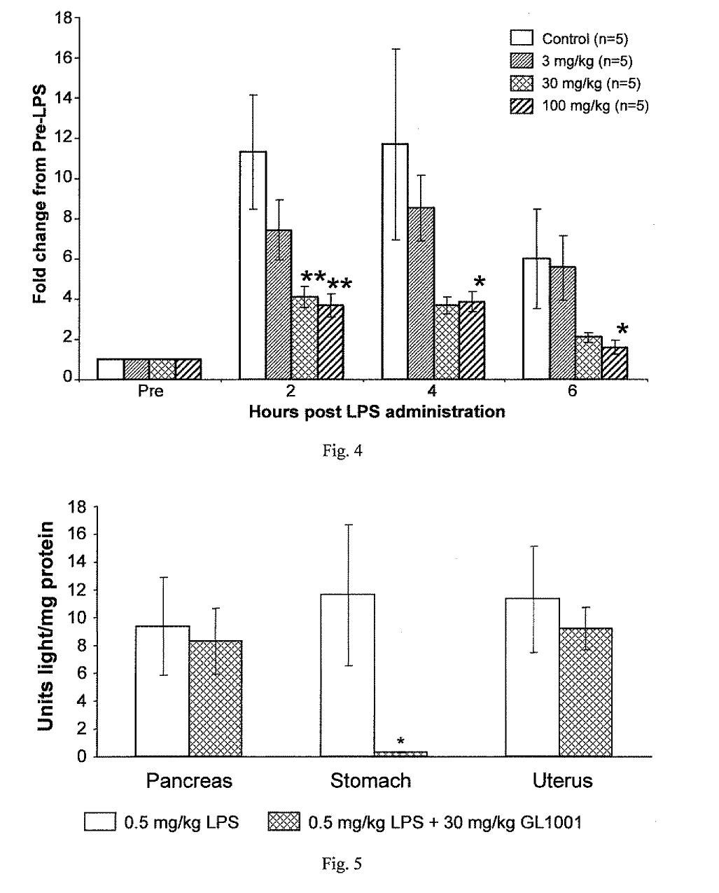

Whole-body imaging showed that GL1001 significantly inhibited LPS-induced in vivo levels of NF-κB-dependent transcription of the luciferase reporter gene, again primarily in the abdominal region. As shown by the quantitative imaging data in FIG. 3, LPS induced a strong NF-κB-dependent luciferase signal in control mice, indicating a strong NF-κB signaling response, as expected. In contrast, mice that were pretreated with GL1001 showed a significantly reduced LPS induced NF-κB signaling response, which could be measured quantitatively in the abdominal region. As inhibition of NF-κB-dependent luciferase activity was observed over the entire dose range of GL1001 in this experiment (30, 100 and 300 mg/kg), the experiment was repeated using a slightly lower dose range (3-100 mg/kg). As shown in FIG. 4, in this lower dose range, GL1001 significantly reduced LPS-induced NF-κB signaling at 30 and 100 mg/kg. These results show that systemic (subcutaneous) administration of the ACE2 inhibitor GL0101 showed significant in vivo anti-inflammatory activity, predominantly in the abdominal region, against bacterial LPS-induced NF-κB-dependent transcription as well as against basal NF-κB-dependent transcription.

Examination of selected organs extracted from NF-κB:Luc mice treated with 0.5 mg/kg LPS and GL1001 at 30 mg/kg or with 0.5 mg/kg LPS alone (FIG. 5) showed a significant (about 37-fold) reduction of LPS-induced NF-κB-dependent transcription in stomachs of GL1001 treated mice, compared to mice treated with LPS alone, but no statistically significant decrease in LPS-induced NF-κB signaling in pancreas and uterus, or in any other organ or organ part that was analyzed (data not shown), namely, liver, kidney, spleen, small intestine, large intestine (colon), mesenteric lymph nodes, cecum (first part of the colon after the small intestine), ovary, uterus, submandibular lymph nodes, brain, heart and lung.

GL1001 inhibition of LPS-induced NF-κB activity in the mouse stomach is consistent with the present observation (above) of ACE2 mRNA expression in normal stomach tissue of human subjects, and with the report of ACE2 mRNA expression in the mouse stomach by Gembardt et al. (2005), supra. The fact that no inhibition of LPS-induced NE-κB activity was observed in other murine tissues previously reported to express high levels of ACE2 mRNA (e.g., kidney, small intestine or colon; see Gembardt et al. (2005), supra) shows that the inhibitory effect on LPS induced NF-κB signaling predominantly in the abdominal region following systemic (subcutaneous) administration of GL1001 is primarily due to some activity of this ACE2 inhibitor in the stomach.

### Example 4

### GL1001 Promotes Gastric Ulcer Healing in a Rat Model

A study was conducted to determine whether GL1001 would promote gastric ulcer healing in a rat model (Wallace et al. (2007) FASEB J. 21:4070-4076). Under halothane anesthesia, acetic acid was applied to a defined area (about 60 mm²) on the serosal surface of the stomach of male Wistar rats. Three days later, a subgroup of 6 rats (Group HH) was sacrificed and the area of the gastric ulcers that had formed was measured planimetrically. The remainder of the rats were divided into treatment groups and received treatments (see below) for 7 days. At the end of that period the rats were sacrificed and the area of the gastric ulcers was measured planimetrically. In addition to testing GL1001, the effects of vehicle and of a positive control (PGF₂ 100 μg/kg) were examined. Each group consisted of 6 rats.

The treatment groups were as follows:

AA: vehicle (5% hydroxypropyl β-cyclodextrin) subcutaneously twice daily.

BB: GL1001 150 mg/kg subcutaneously twice daily.

CC: GL1001 450 mg/kg subcutaneously twice-daily.

DD: vehicle (0.1M citrate buffer) in drinking water.

EE: GL1001 400 mg/kg/day in drinking water.

FF: GL1001 1200 mg/kg/day in the drinking water.

GG: PGE₂ 100 μg/kg orally twice daily.

Serosal application of acetic acid reproducibly induced ulcers in the stomach. By the third day after application of acetic acid, the mean ulcer area was approximately 115 mm². In rats treated twice daily with vehicle subcutaneously or in drinking water for the following 7 days, ulcer healing was apparent (mean ulcer areas of 42±5 mm² and 49±13 mm² respectively; see Table 3). Twice-daily subcutaneous treatment with GL1001 at a dose of 150 mg/kg, but not at a dose of 450 mg/kg, significantly enhanced healing of the ulcers (Group BB vs. Group AA; p<0.05). Significant enhancement of ulcer healing was also observed in rats given twice-daily oral treatment with PGE₂ (Group GG vs. Group AA; p<0.05).

In these experiments, administration of GL1001 via drinking water came close to achieving the targeted doses on days 5 through 10 after ulcer induction. Thus, in the groups that should have received 400 and 1200 mg/kg/day, the actual dose delivered averaged approximately 450 and 1450 mg/kg/day.

TABLE 3
Results of gastric ulcer healing study

| Group | Gastric ulcer area (mm²) |
|---|---|
| AA | 41.7 ± 4.6 |
| BB | 29.0 ± 4.2 |
| CC | 35.2 ± 4.3 |
| DD | 49.3 ± 12.7 |
| EE | 53.1 ± 8.5 |
| FF | 50.1 ± 4.7 |
| GG | 30.5 ± 6.8 |
| HH | 114.8 ± 13.2 |

Significant (p<0.05) healing of ulcers was observed in all groups examined at day 10 as compared to the group sacrificed at day 3 (Group HH). A significant beneficial effect of treatment with PGE₂ (i.e., a significant reduction in ulcer area as compared to the rats treated with vehicle) was observed. Twice-daily treatment with GL1001 subcutaneously at a dose of 150 mg/kg significantly reduced gastric ulcer area as compared to the corresponding vehicle-treated group, but no significant effect was seen with GL1001 treatment at 450 mg/kg.

When administered via drinking water, GL1001 did not significantly affect ulcer healing as compared to vehicle-treated rats. Unlike the acute NSAID study (Example 6 below), the delivery of GL1001 in drinking water in the ulcer healing study did come close to delivering the targeted doses.

### Example 5

### GL1001 Inhibits Acute Indomethacin-Induced Gastric Toxicity in a Rat Model

A study was conducted to determine whether GL1001 would inhibit acute NSAID (indomethacin)-induced gastric toxicity in a rat model (Wallace et al. (1998) Gastroenterol. 115:101-109). Several modes of treatment with GL1001 or vehicle were employed, which included administration via drinking water, oral administration and subcutaneous administration as shown in Table 4. Irrespective of the route of drug or vehicle administration, male Wistar rats were deprived of food for 16 h prior to oral administration of 20 mg/kg indomethacin (Sigma) or vehicle (5% w/v sodium bicarbonate; 1 ml/kg). Three hours later the rats were sacrificed and the severity of gastric damage was blindly scored. This involved measurement of the lengths (in mm) of all hemorrhagic erosions and summing these to give a “gastric damage score” for each stomach. Samples of the stomach were taken for measurement of prostaglandin E₂ (PGE₂) synthesis as described by Wallace et al. (1998), supra, and myeloperoxidase activity as described by Souza et al. (2003) Am. J. Physiol. 285:G54-61. Myeloperoxidase is an enzyme found in all cells of mycloid origin, but particularly enriched within neutrophils. It is therefore used as a biochemical marker of granulocyte infiltration into tissue. Each group consisted of 6 rats. As a negative control, one group received vehicle pretreatments (in the drinking water and by oral gavage) and then received vehicle instead of indomethacin. A positive control group received 100 μg/kg PGE₂ (Cayman Chemical) orally 30 minutes prior to administration of indomethacin, since this has previously been shown to markedly reduce the severity of indomethacin-induced gastric damage (see for example Robert et al. (1979) Gastroenterol. 77:761-767).

TABLE 4
Treatment details for acute indomethacin-induced gastric toxicity study

| | GL1001, PGE₂ or vehicle | | | Indomethacin or |
|---|---|---|---|---|
| Group | p.o. | s.c. | d.w. | vehicle |
| A | — | — | GL1001 | indomethacin |
| B | — | — | V1 | indomethacin |
| C | GL1001 | — | GL1001 | indomethacin |
| D | V2 | — | V1 | indomethacin |
| E | V2 | — | V1 | V3 |
| F | — | GL1001 | GL1001 | indomethacin |
| G | — | V2 | V1 | indomethacin |
| H | GL1001 | — | — | indomethacin |
| I | V2 | — | — | indomethacin |
| J | PGE₂ | — | — | indomethacin |

p.o. = per os; s.c. = subcutaneous; d.w. = in drinking water
V1 = 0.1M citrate buffer
V2 = 15% hydroxypropyl β-cyclodextrin
V3 = 5% sodium bicarbonate

In the experiments where GL1001 (or vehicle) was added to drinking water, the rats were provided with the modified drinking water for 2 days prior to administration of indomethacin (or vehicle). This included the period when the rats were deprived of food. GL1001 was prepared in 0.1M citrate buffer before being added to the drinking water. Based on average water consumption by rats, this was aimed at providing a daily dose of GL1100 of 1200 mg/kg (water consumption was monitored for several days prior to initiation of the study). Oral or subcutaneous administration of GL1001 (or vehicle) was performed 30 minutes prior to administration of indomethacin.

Data are expressed as mean±SEM. When two groups of data were compared, a Student's t-test was used. When more than two groups of data were compared, a one-way analysis of variance followed by the Newman-Keuls test was used. An associated probability of less than 5% was considered statistically significant.

Oral administration of indomethacin resulted in formation of hemorrhagic erosions in the corpus of the stomach. Depending on the route of administration of vehicle prior to indomethacin administration, mean gastric damage scores varied from 20 to 54 (Table 5). Administration of GL1001 both in drinking water and by oral gavage resulted in a significant reduction of severity of indomethacin-induced gastric damage (Group C vs. Group D; p<0.01). It should be noted that the amount of consumption of water by the rats receiving GL1001 in drinking water fell below what was anticipated, so the actual amount of the drug that was delivered was only 500-750 mg/kg/day rather than 1200 mg/kg/day as planned. On the first day of treatment, the reduction in water consumption was likely due to taste aversion, as normal water consumption was observed in the groups receiving vehicle-supplemented drinking water. On the second day, the reduced water consumption was likely a consequence of the rats being fasted, as groups receiving vehicle- and GL1001-supplemented drinking water both consumed less than usual.

Oral pretreatment with GL1001 significantly reduced the extent of indomethacin-induced gastric damage (Group H vs. Group I; p<0.01). Treatment with GL1001 in drinking water alone or subcutaneously did not significantly affect the extent of indomethacin-induced gastric damage as compared to the relevant vehicle-treated controls. Oral treatment with PGE₂ significantly reduced indomethacin-induced gastric damage (Group J vs. Group I; p<0.001).

Gastric PGE₂ synthesis in rats treated only with vehicle (Group E) averaged 82 ng/mg tissue. Administration of indomethacin, irrespective of pretreatment with vehicle, GL1001 or PGE₂, reduced gastric PGE₂ synthesis by >92%. There were no significant differences among the various pretreatments in gastric PGE₂ synthesis.

Myeloperoxidase (MPO) activity in the stomach of rats treated only with vehicle (Group E) averaged 24±4 U/mg tissue. Oral treatment with GL1001 significantly reduced MPO activity as compared to the corresponding vehicle-treated group (Group H vs. Group I, p<0.05). The combination of GL1001 in drinking water and administered subcutaneously also significantly reduced gastric MPO activity (Group F vs. Group G, p<0.01), but treatment with GL1001 only in drinking water (Group A) or by the combination of oral gavage and drinking water (Group C) did not produce a statistically significant reduction of gastric MPO activity (in the latter case, the P value was very close to the threshold for “significance”, p=0.060).

TABLE 5
Results of acute indomethacin-induced gastric toxicity study

| | Gastric damage | Gastric PGE₂ synthesis | Gastric MPO |
|---|---|---|---|
| Group | score | (ng/mg) | activity (U/mg) |
| A | 23.3 ± 8.9 | 5.31 ± 1.54 | 28.4 ± 5.2 |
| B | 20.3 ± 7.1 | 3.83 ± 0.98 | 22.8 ± 3.6 |
| C | 5.2 ± 2.4 | 2.37 ± 0.51 | 13.8 ± 2.7 |
| D | 32.2 ± 7.4 | 3.07 ± 0.55 | 25.1 ± 4.6 |
| E | 0.5 ± 0.5 | 82.16 ± 16.49 | 23.6 ± 3.5 |
| F | 25.5 ± 9.5 | 3.57 ± 0.59 | 12.9 ± 1.4 |
| G | 24.7 ± 7.5 | 3.60 ± 0.32 | 32.3 ± 4.8 |
| H | 20.7 ± 6.3 | 4.44 ± 0.40 | 19.6 ± 2.9 |
| I | 53.5 ± 5.6 | 6.08 ± 1.00 | 29.5 ± 2.8 |
| J | 6.2 ± 3.1 | 6.10 ± 1.00 | 22.8 ± 2.2 |

In this study, indomethacin produced extensive gastric damage in all vehicle-pretreated groups. Pretreatment with PGE₂, the positive control, produced a significant reduction in the extent of indomethacin-induced gastric damage. Pretreatment with GL1001 also produced a significant reduction of the severity of indomethacin-induced gastric damage, an effect that was dependent on the route of administration. In both circumstances in which GL1001 was administered orally (i.e., alone or also via drinking water), this drug was effective. No significant effect was seen when it was administered in the drinking water alone or subcutaneously+drinking water. It is possible that the effect seen with oral administration but not by other routes of administration is a result of a higher concentration of GL1001 making contact with the mucosal surface (i.e., either a topical effect of the drug, or a higher concentration achieved in the gastric mucosa). As the solution of GL1001 for oral administration had a pH of about 12, it is also possible that the beneficial effects of this drug were related to a buffering of luminal acid. Profound suppression of acid secretion can reduce the severity and incidence of NSAID-induced gastric damage. See, for example, Taha et al. (1996) New Eng. J. Med. 334:1435-1439; Scheiman et al. (2006) Am. J. Gastroenterol. 101:701-710.

### Example 6

### GL1001 Inhibits Acute Diclofenac-Induced Gastric Toxicity in a Rat Model

A study was conducted to determine whether GL1001 would inhibit acute NSAID-induced gastric toxicity in a rat model (Wallace et al. (1998), supra), using diclofenac, a more clinically important NSAID than the indomethacin used in Example 6 above.

Male Wistar rats were deprived of food but not water for 16 hours prior to oral administration of GL1001 90 mg/kg, incubated for 30 minutes and then given oral diclofenac 50 mg/kg. Each group consisted of 6 rats. As a negative control, one group received vehicle pretreatment, and then received vehicle instead of diclofenac. A positive control group received PGE₂ 100 μg/kg orally, 30 minutes prior to administration of diclofenac. Treatments are summarized in Table 6. All treatments were administered by oral gavage.

Three hours after diclofenac treatment, all animals were sacrificed and stomachs were removed and imaged to determine the severity of gastric damage. Additionally, stomach tissue samples were taken to assess disease activity and pharmacodynamic effects. Gastric damage scores were determined from photographic images (Ma & Wallace (2003) Am. J. Physiol. Gastrointest. Liver Physiol. 279; 341-346). Removed stomachs were opened along the greater curvature, pinned out on a wax block, and photographed along a 25 mm ruler reference with a digital 35 mm camera. The lesion area was then determined using enlarged images and computer software (www.scioncorp.co/pages/scion_image_windows.htm) that allows superimposed drawing of the perimeter of a wound and calculation of the area based on the ruler reference. Stomach samples (excluding forestomach) were used for measurements of PGE₂ concentration, MPO activity and ACE2 activity. PRE₂ determinations are used in this study as a hiomarker of diclofenac effect since its mechanism of action involves inhibition of cyclooxygenase (COX-2 and COX-1) enzymes which in turn results in inhibition of PGE₂ synthesis. Concentration of PGE₂ from stomach tissue samples was performed as described by Souza et al. (2003), supra, and quantification was obtained by LC-MS. MTO, an enzyme found in abundance in granulocytes, is used as a biochemical marker of tissue inflammation. When tissues undergo inflammatory processes, granulocytes are recruited to the site of inflammation and infiltrate the affected tissue giving rise to increase MPO activity (Souza et al. (2003), supra). Last, the extent of inhibition of ACE2 activity was determined to establish whether GL1001 was effective in inhibiting its ACE2 target in stomach tissues. The ACE2 activity assay is similar to the method of Vickers et al. (2002) J. Biol. Chem. 277:14838-14843, wherein purified recombinant ACE2 (100 pM) and a quenched fluorescent substrate (100-150 μM APK (7-methoxycoumarin-4-yl)acetyl-Ala-Pro-Lys (2,4-dinitrophenyl)) are used in end-point fluorescence reactions (excitation 320 nm, emission 405 nm). For stomach ACE2 measurements, tissues were homogenized in the presence of a general protease inhibitor (PMSF) and specific ACE inhibitors. PGE₂ was obtained from Sigma, MPO activity kit from CytoStore and purified rACE and substrate from R&D Systems.

TABLE 6
Treatment details for acute diclofenac-induced gastric toxicity study

| | GL1001, PGE₂ | Diclofenac or |
|---|---|---|
| Group | or vehicle | vehicle |
| A | V2 | V4 |
| B | V2 | diclofenac in V4 |
| C | GL1001 in V2 | diclofenac in V4 |
| D | PGE₂ in V4 | diclofenac in V4 |

V2 = 15% hydroxypropyl β-cyclodextrin (pH 5; final pH 11-12 with GL1001)
V4 = 1% carboxymethylcellulose (pH 5; final pH ~8 with diclofenac)

Data are expressed as means with standard errors. Student's t-test was applied to the data, which conformed to normal distribution and equal variance.

Oral administration of diclofenac resulted in the formation of hemorrhagic lesions and ulcers in the corpus of the stomach, with a mean gastric damage score of 17.8 (Table 7). When animals were pretreated with GL1001 by oral gavage, a mean gastric damage score of 2.6 was observed, corresponding to a significant reduction of the severity of diclofenac-induced gastric damage as compared to the vehicle-diclofenac group (Group C vs. Group B, p<0.02). In the case of oral pretreatment with PCGE₂ positive control), a complete reduction of diclofenac-induced gastric damage was observed to the point of total absence of gastric lesions, similar to the observed lack of erosions in the vehicle/vehicle control animals. These results confirm that GL1001 is effective in providing gastroprotection from acute gastric damage induced by the NSAID diclofenac.

In order to confirm effectiveness of diclofenac in all groups that received it, independently of gastric damage, gastric PGE₂ levels were quantified. The vehicle control group (Group A), the only group not treated with diclofenac, had an average PGE₂ concentration in stomach tissue of 43.22 pg/mg. In contrast all treatment groups that received diclofenac had no detectable PGE₂ levels, indicating a significant decrease in PGE₂ synthesis.

MPO activity in gastric tissues could not be reliably determined in this study due to low levels of the enzyme in all groups. The signal/noise ratio of the assay was too large to ascribe any significance to MPO activity measurements (data not shown).

ACE2 activity was measured to determine the extent of inhibition of ACE2 in stomach tissues. The average activity of ACE2 in normal gastric tissue of naïve animals was 772 pmoles/mg, as shown by the vehicle control group (Group A). Activity of ACE2 showed a trend towards up-regulation in diclofenac-treated animals (Group B vs. Group A, p=0.052). In GL1001-dosed rats, ACE2 activity was significantly reduced to 436 pmoles/mg (Group C vs. Group B, p<0.0001).

TABLE 7
Results of acute diclofenac-induced gastric toxicity study

| | Gastric damage | Gastric PGE₂ | Gastric ACE2 |
|---|---|---|---|
| Group | score | concentration (pg/mg) | activity (pmol/mg) |
| A | 0 | 43.22 ± 12.13 | 772 ± 30 |
| B | 17.80 ± 5.08 | 0 | 880 ± 39 |
| C | 2.63 ± 0.87 | 0 | 436 ± 20 |
| D | 0 | 0 | 688 ± 30 |

When tissue homogenates are used to determine ACE2 activity, as in this study, the fluorescence signal in the ACE2 assay could come from ACE2 activity, which cleaves the last residue of the quenched fluorescent tripeptide substrate used in the assay, from background fluorescence or from cleavage of the tripeptide substrate by proteases other than ACE2. Consequently, in order to be able to ascribe ACE2 specific activity to the observed fluorescence signal, we determined the extent of GL1001-inhibitable signal in the tissue samples. Duplicate tissue samples corresponding to Groups A, B and C were either incubated (“spiked”) with a vast excess of GL1001 (>IC₉₀) or not spiked. It was found that spiking with GL1001 inhibited ACE2 activity in tissues of Groups A and B (control and diclofenac-treated respectively) as observed by a decrease of 40-50% in the fluorescence signal. This 40-50% fraction corresponds to ACE2 specific activity. In contrast, the samples from Group C (the group dosed with GL1001) showed no further inhibition of ACE2 as observed by no further decrease in the fluorescence signal. These results indicate that in vivo dosed GL1001 effectively inhibits ACE2 in stomach tissues, and that when GL1001 is orally dosed at 900 mg/kg in the conditions of this study, the amount of compound that reaches the stomach is equal to or greater than the amount needed for full inhibition of stomach ACE2 activity.

Taken together, these results show that GL1001 is effective in reducing the severity of diclofenac-induced gastric damage with concomitant inhibition of ACE2 activity, and further suggest existence of a target-mediated gastroprotection mechanism independent of restoration of PGE₂ levels. An anti-inflammatory mechanism of gastroprotection would be consistent with these observations.

All patents and publications cited herein are incorporated by reference into this application in their entirety.

The words “comprise”, “comprises”, and “comprising” are to be interpreted inclusively rather than exclusively.

What is claimed is:

1. A method for managing or treating an inflammatory, erosive, dyspeptic or reflux disorder of the proximal digestive tract, said disorder comprising a condition other than chronic gastritis or Crohn's disease, the method comprising administering to a subject having said disorder a therapeutically effective amount of an ACE2 inhibitor.

2. The method of claim 1, wherein the ACE2 inhibitor exhibits in vitro an ACE2IC₅₀ and/or an ACE2 Ki not greater than about 1000 mM.

3. The method of claim 1, wherein the ACE2 inhibitor exhibits selectivity for ACE2 versus ACE, as expressed by the ratio of IC₅₀(ACE) to IC₅₀(ACE2), of at least about 10³.

4. A method for managing or treating an inflammatory, erosive, dyspeptic or reflux disorder of the proximal digestive tract, said disorder comprising a condition other than chronic gastritis or Crohn's disease, the method comprising administering to a subject having said disorder a therapeutically effective amount of a compound of formula 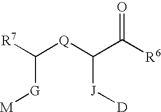
wherein
R⁶ is hydroxyl or a protecting prodrug moiety;
R⁷ is hydrogen, carboxylic acid, ether, alkoxy, an amide, a protecting prodrug moiety, hydroxyl, thiol, heterocyclyl, alkyl or anine;
Q is CH₂, O, NH or NR³, wherein R³ is substituted or unsubstituted C₁₋₅ branched or straight chain alkyl, C₂₋₅ branched or straight chain alkenyl, substituted or unsubstituted acyl, aryl or a C₃₋₈ ring;
G is a covalent bond or a CH₂, ether, thioether, amine or carbonyl linking moiety;
M is heteroaryl, substituted with at least one subanchor moiety comprising a substituted or unsubstituted cycloalkyl or aryl ring, linked thereto through a sublinking moiety (CH₂)ₙ or (CH₂)ₙO(CH₂), where n is an integer from 0 to 3;
J is a bond or a substituted or unsubstituted alkyl, alkenyl or alkynyl moiety; and
D is alkyl, alkenyl, alkynyl, aryl or heteroaryl, optionally linked to G or M to form a ring;
or a pharmaceutically acceptable salt or prodrug thereof.

5. The method of claim 4, wherein, in the formula for the compound, R⁶ is hydroxyl;
R⁷ is carboxylic acid;
Q is NH;
G is CH₂;
the heteroaryl group of M is imidazolyl, thienyl, triazolyl, pyrazolyl or thiazolyl; and the subanchor moiety is C₃₋₄ cycloalkyl, phenyl, methylenedioxyphenyl, naphthalenyl, or phenyl having 1 to 3 substituents independently selected from halo, C₁₋₆ alkyl, C₃₋₆ cycloalkyl, trifluoromethyl, Cab alkoxy, trifluoromethoxy, phenyl, cyano, nitro and carboxylic acid groups, and is linked to the heteroaryl group through a (CH₂)ₙ or (CH₂)O(C₁H₂) sublinking moiety, where n is an integer from 0 to 3;
J is a bond or CH₂ moiety; and
D is C₁₋₆ alkyl, C₃₋₄ cycloalkyl or phenyl.

6. The method of claim 4, wherein the compound is (S,S)-2-[1-carboxy-2-[3-(3,5-dichlorobenzyl)-3H-imidazol-4-yl]-ethylamino]-4-methylpentanoic acid or a pharmaceutically acceptable salt or prodrug thereof.

7. The method of claim 4, wherein the disorder comprises at least one condition selected from the group consisting of duodenitis, acute gastritis, esophagitis, acute peptic ulcer, celiac sprue, functional dyspepsia and gastroesophageal reflux disease.

8. The method of claim 4, wherein the disorder is an acute inflammatory or erosive disorder of one or more of the duodenum, stomach and esophagus.

9. The method of claim 8, wherein said managing or treating comprises promotion of ulcer healing.

10. The method of claim 4, wherein the disorder comprises alcohol- or medication-induced acute peptic ulcer or dyspepsia.

11. The method of claim 4, wherein the disorder comprises NSAID-induced acute peptic ulcer.

12. The method of claim 4, wherein the disorder comprises functional dyspepsia manifesting as one or more of heartburn, eructation, early satiety, dysphagia, bloating and nausea.

13. The method of claim 4, wherein the disorder comprises gastroesophageal reflux disease.

14. The method of claim 4, wherein the disorder is refractory to proton pump inhibitors, H₂ receptor blockers or both.

15. The method of claim 4, wherein said therapeutically effective amount comprises a dosage amount of the compound of about 0.5 to about 5000 mg/day.

16. A method for protecting from erosion or ulceration a mucosal surface of the proximal digestive tract, comprising administering to a subject at risk for said erosion or ulceration a therapeutically effective amount of a gastroprotective agent comprising
(a) an ACE2 inhibitor; or
(b) a compound of formula 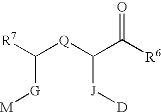
wherein
R⁶ is hydroxyl or a protecting prodrug moiety;
R⁷ is hydrogen, carboxylic acid, ether, alkoxy, an amide, a protecting prodrug moiety, hydroxyl, thiol, heterocyclyl, alkyl or amine;
Q is CH₂, O, NH or NR¹, wherein R³ is substituted or unsubstituted C₁₋₅ branched or straight chain alkyl, C₂₋₅ branched or straight chain alkenyl, substituted or unsubstituted acyl, aryl or a C₃₋₈ ring;
G is a covalent bond or a CH₂, ether, thioether, amine or carbonyl linking moiety;
M is heteroaryl, substituted with at least one subanchor moiety comprising a substituted or unsubstituted cycloalkyl or aryl ring, linked thereto through a sublinking moiety (C₁H₂)ₙ or (CH₂)ₙO(CH₂)ₙ where n is an integer from 0 to 3;
J is a bond or a substituted or unsubstantiated alkyl alkenyl or alkynyl moiety; and
D is alkyl, alkenyl, alkynyl, aryl or heteroaryl, optionally linked to G or M to form a ring;
or a pharmaceutically acceptable salt or prodrug thereof.

17. The method of claim 16, that provides protection from duodenal, gastric and/or esophageal ulcer formation, development or recurrence related to concomitantly administered medication.

18. The method of claim 17, wherein the concomitantly administered medication comprises an NSAID.

19. The method of claim 16, wherein the gastroprotective agent comprises an ACE2 inhibitor exhibiting in vitro an ACE2IC₅₀ and/or an ACE2 Ki not greater than about 1000 nM.

20. The method of claim 16, wherein the gastroprotective agent comprises an ACE2 inhibitor exhibiting selectivity for ACE2 versus ACE, as expressed by the ratio of IC₅₀(ACE) to IC₅₀(ACE2), of at least about 10³.

21. The method of claim 16, wherein the gastroprotective agent comprises (S,S)-2-[1-carboxy-2-[3-(3,5-dichlorobenzyl)-3H-imidazol-4-yl]-ethylamino]-4-methylpentanoic acid or a pharmaceutically acceptable salt or prodrug thereof.

22. The method of claim 21, wherein the (S,S)-2-[1-carboxy-2-[3-(3,5-dichlorobenzyl)-3H-imidazol-4-yl]-ethylamino]-4-methylpentanoic acid or salt or prodrug thereof is administered in a dosage amount of about 0.5 to about 5000 mg/day.

23. A therapeutic combination comprising at least one NSAID in an anti-inflammatory, analgesic or antipyretic effective amount and a gastroprotective agent in an amount effective to protect mucosal surfaces of the proximal digestive tract from NSAID-induced erosion or ulceration; wherein the gastroprotective agent comprises
(a) an ACE2 inhibitor; or
(b) a compound of formula 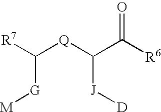
wherein
R⁶ is hydroxyl or a protecting prodrug moiety;
R⁷ is hydrogen, carboxylic acid, ether, alkoxy, an amide, a protecting prodrug moiety, hydroxyl, thiol, heterocyclyl, alkyl or amine;
Q is CH₂, O, NH or NR³, wherein R³ is substituted or unsubstituted C₁₋₅ branched or straight chain alkyl, C₂₋₅ branched or straight chain alkenyl, substituted or unsubstituted acyl, aryl or a C₃₋₈ ring;
G is a covalent bond or a CH₂, ether, tiloether, amine or carbonyl lining moiety;
M is heteroaryl, substituted with at least one subanchor moiety comprising a substituted or unsubstituted cycloalkyl or aryl ring, linked thereto through a sublinking moiety (C117)ₙ or (CH₂)ₙO(CH₂)ₙ where n is an integer from 0 to 3;
J is a bond or a substituted or unsubstituted alkyl, alkenyl or alkynyl moiety; and
D is alkyl alkenyl, alkynyl, aryl or heteroaryl, optionally linked to G or M to form a ring;
or a pharmaceutically acceptable salt or prodrug thereof.

24. The combination of claim 23, wherein the at least one NSAID and the gastroprotective agent are present in a single dosage form.

25. The combination of claim 23, wherein the at least one NSAID is selected from the group consisting of aspirin, indomethacin, diclofenac, ibuprofen, naproxen, oxaprozin, meloxicam and celecoxib, and the gastroprotective agent comprises (S,S)-2-[1-carboxy-2-[3-(3,5-dichlorobenzyl)-3H-imidazol-4-yl]-ethylamino]-4-methylpentanoic acid or a pharmaceutically acceptable salt or prodrug thereof.
